(19) United States
(12) Patent Application Publication
(10) Pub. No.: US 2023/0330052 A1
(43) Pub. Date: Oct. 19, 2023

(54) COMPOSITION OF ACTIVE AGENTS TO POSITIVELY AFFECT A ROBUST MAMMALIAN ENDOCANNABINOID SYSTEM TONE TO BETTER ADDRESS AGE RELATED DISCOMFORT

(71) Applicant: Compact Disc Incorporated, Silver Spring, MD (US)

(72) Inventors: Robert RAGER, Silver Spring, MD (US); Tama COPEMAN, Emmaus, PA (US); Zachary PHILLIPS, Washington D.C., DC (US)

(21) Appl. No.: 18/214,137

(22) Filed: Jun. 26, 2023

Related U.S. Application Data
(63) Continuation-in-part of application No. 17/191,996, filed on Mar. 4, 2021, now Pat. No. 11,684,600.
(60) Provisional application No. 63/100,221, filed on Mar. 4, 2020.

(51) Int. Cl. A61K 31/202; A61K 31/015; A61K 9/16; A61K 31/12; A61K 31/165; A61K 9/10; A61K 9/00; A61K 31/00

(52) U.S. Cl. CPC A61K 31/202; A61K 31/015; A61K 9/16; A61K 31/12; A61K 31/165; A61K 9/10; A61K 9/0014; A61K 31/658

(57) ABSTRACT

Compositions and methods for prophylactically sustaining and/or maintaining a robust endocannabinoid system in patients for modulating excess inflammatory response and inflammatory diseases over the mammalian lifespan to mitigate age-related discomfort. Specifically, a method of uniquely treating inflammatory and neuropathic pain in a mammal by administering a synergistic pharmaceutical daily dosage form comprising: palmitoylethanolamide (PEA), beta-caryophyllene (BCP) and docosahexaenoic acid (DHA).

### RELATED DISCOMFORT

This application claims priority to U.S. Provisional Patent Application Ser. No. 63/100,221, filed on Mar. 4, 2020, and U.S. patent application Ser. No. 17/191,996, filed on Mar. 4, 2021, the contents of each of which are incorporated herein by reference in their entirety.

### BACKGROUND

### Field

The present subject matter relates to compositions and methods for prophylactically sustaining and/or maintaining a robust endocannabinoid system (eCS) in patients for modulating excess inflammatory response and inflammatory diseases over the mammalian lifespan to mitigate age-related discomfort.

### Description of the Related Art

Persistent pain in neuropathic conditions is often refractory to conventional analgesic therapy, providing most patients with only partial, if any, symptomatic relief.1 Chronic pain is a frequent condition calling for improved analgesics, and it affects an estimated 20% of people worldwide.2, 3 Treatment of such complex pain with one or a combination of two analgesics at the most is typical, although generally inadequate.4, 5

It seems prudent to make use of endogenous repair and homeostasis mechanisms, already available in the body. Cannabinoids, classified as locally produced and locally acting autacoids, are scientifically recognized as natural anti-inflammatory compounds with superior efficacy versus NSAIDs (nonsteroidal anti-inflammatory drugs) that decrease pain6 and lower fever, and, in higher doses, decrease pain but without well-known negative side effects of long-term use. These include risk of serious gastrointestinal side effects such as perforation, ulceration, or bleeding.7, 8 Current chronic pain treatments have numerous shortcomings. Tolerability, addiction and overdose potential, and serious adverse effects often limit its use over an extended period, and the pain control achieved by such drugs are often not ideal.7 Even the effects of often prescribed drugs such as pregabalin or opioids are disappointing for patients suffering from neuropathic pain. Furthermore, some classes of analgesics might decrease pain, but can also negatively interfere with our body repair systems, such as the non-steroidal anti-inflammatory drugs (NSAIDs) including cyclooxygenase (COX) inhibitors.

While it is widely known these last compounds inhibit pro-inflammatory cascades in many chronic pain states, much less it is known that the same inhibition impairs the biological activity of endogenous repairing and protecting molecules, such as the lipoxins and the resolvins with “pro-resolving” properties, representative of the temporal cellular and biochemical events in the onset and resolution of inflammation.9, 10 The early phase of inflammation is characterized by the release of pro-inflammatory mediators and extravascular accumulation of neutrophils, followed by infiltration of monocytes that differentiate into macrophages. This phase is characterized by the formation of anti-inflammatory and pro-resolution mediators (e.g., lipoxins [LXs] and resolvins). These mediators stop further neutrophil trafficking and facilitate the removal of inflammatory debris such as apoptotic cells. The ingestion of apoptotic cells results in potent anti-inflammatory effects through the production of anti-inflammatory cytokines such as TGF-β1, IL-10 and PGE2, and the decreased release of pro-inflammatory mediators, including IL-8, TNF-α and TXA2.11

### Taming Inflammation Requires Resolution and a Robust eCS

The resolution of inflammation is an active process controlled by endogenous mediators with selective actions on neutrophils and monocytes. The initial phase of the acute inflammatory response is characterized by the production of pro-inflammatory mediators followed by a second phase in which lipid mediators with pro-resolution activities may be generated. The identification of these mediators has provided evidence for the dynamic regulation of the resolution of inflammation. Among these endogenous local mediators of resolution, LXs, lipid mediators typically formed during cell-cell interaction, were the first to be recognized. More recently, families of endogenous chemical mediators, termed resolvins and protectins, were discovered.12

LXs and aspirin triggered LXs are considered to function as ‘braking signals’ in inflammation, limiting the trafficking of leukocytes to the inflammatory site. LXs are actively involved in the resolution of inflammation-stimulating non-phlogistic phagocytosis of apoptotic cells by macrophages. Furthermore, LXs have emerged as potential anti-fibrotic mediators that may influence pro-fibrotic cytokines and matrix-associated gene expression in response to growth factors.10 Our composition of matter includes a key component, DHA Omega 3, giving rise to resolvins and related compounds (e.g., protectins) through pathways involving cyclooxygenase and lipoxygenase enzymes to resolve the inflammatory responses.13

NSAIDs inhibit the synthesis of many autacoids—a class of natural compounds which are called into being only as-needed and act as tissue hormones and natural pain relievers, thus NSAIDs do not contribute to resolving chronic inflammation, nor do they repair injury.14 It is not only the NSAIDs negatively influencing inflammatory resolution, opioids are also increasingly recognized as pro-inflammatory compounds. Opioid-induced glial activation and its pro-inflammatory consequences are regarded as factors for failure of analgesia as well as a pathogenetic base for the emergence of opioid tolerance.15, 16 In all these cases analgesics negatively interfere with endogenous autacoid repair and anti-inflammatory processes.6 To find new inroads in the treatment of chronic pain via the autacoids therefore seems a promising way of identifying new analgesic compounds, acting via endogenous healing mechanisms. It seems indeed quite logical to make use of endogenous repair and homeostasis mechanisms, already available in the body. Autacoid Pain Medicine may be a new fundament for the design of the 21st century pain medicine.18

Cannabinoids, particularly endocannabinoids, their cannabinoid receptors and enzymes that construct and deconstruct the neurotransmitter cannabinoids only as needed, comprise the Endocannabinoid System (eCS) are responsible for regulating homeostasis via signaling throughout the central and peripheral nervous systems. Furthermore, cannabinoids interacting with the CB2 receptor are scientifically recognized as natural anti-inflammatory autacoid compounds. These anti-inflammatory cannabinoids uniquely act on the immune system by way of the mammalian endogenous communication system known as eCS22 with superior efficacy versus NSAIDs. Both cannabinoids and NSAIDs are shown to decrease pain6 and lower fever, but cannabinoids achieve this without any well-known negative side effects of long-term use. Long-term risks of NSAID use include serious gastrointestinal side effects, and according to a recent meta-analysis, potential increased risk of coronary heart disease.19, 20

But because of life stresses, both internal and external to living mammals over the lifespan, the endoCannabinoid System (eCS) can become less efficient due to diminished performance of any and/or all the eCS components.23 Diminished eCS performance can lead to an overall disruption of homeostasis and therefor chronic, systemic inflammation, and poor wellness and painful age-related discomforts.23

Diminished homeostasis leads to increased inflammation which can result in damaged tissues, nerves, and joints, often associated with age related discomfort. Chronic conditions of diminished eCS performance from life stresses, illnesses and aging have been described as Clinical Endocannabinoid Deficiency (CECD). Several otherwise unexplained conditions including, but not limited to, migraine, irritable bowel syndrome, autoimmune-mediated maladies, and fibromyalgia have been attributed to CEDC.23, 24, 25

The NIH has patented the Cannabis derived compound CBD (Cannabidiol) for its antioxidant and anti-inflammatory properties.26 Subsequent research has established CBD as an effective anti-inflammatory plant-derived phyto-cannabinoid compound for use in pain mitigation related to tissue and nerve damage typically due to inflammation in mammals. CBD is also known to help sustain a robust body-wide endoCannabinoid (eCB) System Tone (eCS Tone) based on optimal performance of the various components of the “classic” endocannabinoid system (eCS) (i.e., cannabinoid receptors CB1 and CB2, the eCB neurotransmitter ligands ANA and 2-AG, and their metabolic enzymes FAAH or MAGL).27, 28

Cannabis as a genus (i.e., both Marijuana and Hemp plant families) is classified by the US Drug Enforcement Administration (DEA) as a Class 1 Controlled Substance. ‘Marijuana’, the Cannabis plant family with flowers containing naturally significant quantities of psychoactive THC (Tetrahydrocannabinol) and nominal amounts of non-psychoactive CBD. Hemp plants, the Cannabis plant family with strong fibers (legally classified as Industrial Hemp) have naturally significant amounts of CBD but only trace amounts of THC (<0.3% dry weight). As a result of strict DEA classification of all of Cannabis instead of only THC-laden Marijuana, has caused a hopelessly confusing condition regarding CBD legality resulting in tightly controlled limits on CBD research as well as a total lack of federal regulations required to ensure FDA product safety labeling. Because of the many positive wellness related effects of CBD acting on the eCS, CBD has attracted considerable attention and interest from the public.29 The key influence of CBD is on the endogenous signaling process of homeostasis that maintains physiological/biochemical balance necessary for wellness in all living mammals.30

All vertebrate animals have an endocannabinoid system (eCS). In fact, the eCS is ubiquitous in nearly all animals from mammals to the more primitive phyla such as Cnidaria; the early emergence of the eCS in the evolution of the Phyla, indicates its long-standing biological importance.31 The eCS, providing homeostatic balance after various stresses to the nervous, immune, and other organ systems, opened the door to novel approaches for the management and treatment of inflammatory and immune-related disease states.32

The eCS consists of three parts: (1) endogenous ligands, (2) G-protein coupled receptors (GPCRs), and (3) enzymes to construct, degrade and recycle the ligands. Four endogenous cannabinoid molecules have been identified as ligands in the eCS to date: anandamide (ANA or arachidonoylethanolamide), 2-AG (2-arachidonoyl glycerol), OEA (oleoylethanolamide), and PEA (palmitoylethanolamide). Three G-coupled protein receptors have been described (including GPR55—aka CB3), with others being considered (e.g., GPR119).33 Coincidentally, the phytochemicals are produced in large quantities by the Cannabis Sativa L plants (predominantly CBD in Hemp and THC in Marijuana). These plant-based cannabinoids (termed phytocannabinoids) can interact with this system as ligands.31, 34

The eCS is a biological signaling system involved in regulating various physiological and biological balancing processes (homeostasis) in all mammals. Furthermore, the eCS is essential to CNS homeostasis and plays a significant role in the regulation of the inflammatory processes and pain signaling.35 The endocannabinoid system has been shown to have a homeostatic role (i.e., homeostasis, the state of steady internal biochemical balance conditions maintained in various systems present in all healthy mammals) by controlling several metabolic functions, such as energy storage and nutrient transport.35 This dynamic state of equilibrium management concerning the constancy of the internal environment in which the cells of the body live and survive is the condition of optimal functioning for the organism. Equilibrium variables of great importance to proper body function include body temperature, fluid balance, and rates of gas exchange. Other variables include the pH of extracellular fluid, the concentrations of sodium, potassium and calcium ions, as well as that of the blood sugar level all needing to be regulated despite changes in the environment, diet, health, or level of activity.35

Each of these variables is controlled by one or more regulators or homeostatic mechanisms, which together maintain the living organism. Optimum health and wellness depend on robust operational tone, or efficiency, of the eCS to regulate homeostasis throughout the mammalian lifespan. Disruption of homeostasis due to stress, disease, aging, inflammation32 or other conditions of modern life can result in reduced eCS performance or poor eCS Tone, yielding various age-related discomforts in which pain is typically involved.35 Certain painful conditions have been positively associated with Clinical Endocannabinoid Deficiency (CECD). Emerging literature documents CECD as an etiology in migraine, fibromyalgia, irritable bowel syndrome, psychological disorders, and of other illnesses and conditions potentially contributing to various additional age-related discomforts.24

The best-known natural endogenous endocannabinoids are anandamide (ANA), 2 acyl-glycerol (2AG) and palmitoylethanolamide (PEA).36 These naturally produced bioactive lipid autocoids are capable of interacting directly or indirectly with the endocannabinoid receptors such as CB1, CB2, and CB3 (aka GPR55) the activation thereof is responsible for different properties including, but not limited to, anti-inflammatory, antioxidant and analgesic properties. These receptors are sensitive to endogenous cannabinoids, as well as phytocannabinoid and certain related compounds derived from plants and/or cannabinoids of synthetic derivation, which belong to receptors coupled to protein G.37

CB1 and CB2 receptors are structurally similar. CB1 receptors are abundant in the central nervous system, particularly the hippocampus and associated cortical regions, in the brain and in the basal ganglia. CB2 receptors are abundant in the gut and peripheral nervous system mostly associated with the immune system being primarily present in T cells, the mastocytes, B lymphocytes and at the level of the hematopoietic cells as well as in the peripheral nervous terminations, playing a key role in the antinociceptive, antalgic and anti-inflammatory activity. Endocannabinoids of natural origin, including PEA, represent an important alternative to the traditional anti-inflammatory drugs treating inflammation (neuroinflammation or other types of inflammatory conditions) and in all conditions characterized by painful symptomatology.30 PEA's protective effects are mediated by its cellular targets, including direct activation of GPR55 (CB3) and PPAR-a receptors and the indirect activation of cannabinoid receptors CB1 and CB2 and TRPV1 channels.38, 39 CBD and THC are natural phytocannabinoid compounds in Cannabis (plant families commonly known as Marijuana and Hemp). CBD and THC are mammalian CB (cannabinoid) receptor agonists that have been used to reduce pain in humans for millennia. But THC causes ‘intoxication’ and its use and research into its mechanism of action have been severely limited by the Drug Enforcement Administration (DEA) listing of Cannabis, including CBD, as a continuing Class 1 Controlled Substance for many decades.38 Recent studies indicate potential damage to the fetus of pregnant women using CBD, especially inauthentic CBD oils that may contain heavy metals that cannabis is good at extracting from the soil.40

Because, in 2018 CBD became an FDA approved drug (i.e., CBD isolate known as Epidiolex® for pediatric seizures)41, CBD and other Cannabis derived phytocannabinoids (i.e., THC) are currently entangled in State and Federal legal issues, alternative approaches to maintaining robust eCS tone functioning into old age are necessary and desirable.

Palmitoylethanolamide (PEA) is a non-classical endocannabinoid (autocoid) bioactive lipid mediator belonging to the N-acyl-ethanolamine (NAE) fatty acid amide family.42 Synthesized on demand within the lipid bilayer,43 it acts locally44 and is found in all tissues including the brain.45 PEA is thought to be produced as a pro-homeostatic protective response to cellular injury and is usually up-regulated in disease states. Its pleiotropic effects include anti-inflammatory, analgesic, anticonvulsant, antimicrobial, antipyretic, antiepileptic, immunomodulatory and neuroprotective activities.46

PEA's multi-faceted effects are due to its unique mechanisms of action that affect multiple pathways at different sites;44 primarily targeting the nuclear receptor peroxisome proliferator-activated alpha (PPAR-α), PEA also acts on novel cannabinoid receptor, CB3—G protein-coupled receptor 55 (GPR55) and G protein-coupled receptor 19.33, 36, 47, 48, 49 The cannabinoid receptor CB3 plays a role in mechanical hyperalgesia associated with inflammatory and neuropathic pain.50 Moreover, PEA indirectly activates cannabinoid receptors CB1 and CB2 through inhibiting the degradation of the endocannabinoid, anandamide (ANA), a phenomenon known as the “entourage effect”.43

Given the redundancy and complex nature of the underlying pathogenesis, where multi-modal, alternative pathways including a variety of receptors, neurotransmitters and regulatory systems participate in the pain process51, novel approaches involving redundancy of multimodal alternative pathways simultaneously involved in the pain mitigation process, are logically justifiable as a unique approach.52 The present subject matter addresses these needs.

### SUMMARY

This present subject matter addresses a prophylactic approach to mitigating age-related discomfort from increased chronic inflammation due to a weakened endocannabinoid system's inability to robustly regulate homeostasis. Homeostasis is how the mammalian body actively regulates all biological systems to maintain a confined range of optimum conditions—consistently, even during sleep. Homeostasis is typically maintained through negative feedback signals only when something changes, and the body begins to correct itself. The endocannabinoid system (eCS) plays an essential regulatory role in that correction.30

In one embodiment, the present subject matter relates to a method of treating inflammatory and neuropathic pain in a mammal comprising:
A method of helping to replenish levels of endogenous palmitoylethanolamide (PEA) and docosahexaenoic acid (DHA), restoring their protective, anti-inflammatory, and analgesic effects, and protecting the health of a mammal in need thereof, comprising a synergistic supplemental daily dosage form containing exogenous palmitoylethanolamide (PEA) in an ultra-micronized form, docosahexaenoic acid (DHA) and beta-caryophyllene (BCP).

In another embodiment, the present subject matter relates to a method of treating neuropathic and inflammatory pain, comprising: administering to a mammal in need thereof an oral, rectal, topical, or transdermal dosage form, comprising one or more of cream, liquid, lotion, balm, ointment, chew, gummy, patty, freeze-dried food product, cookie, treat, suppository, powder, tablet, wherein the synergistic ingredients comprise:
Wherein palmitoylethanolamide (PEA) is contained in an amount between 25 mg and 1200 mg per dosage, preferably 50 mg to 1000 mg, and more preferably 50 mg to 600 mg, and not exceeding 3600 mg per dose; wherein beta-caryophyllene (BCP) is contained in an amount between 10 mg and 500 mg per dosage, preferably 15 mg to 300 mg, and more preferably 20 mg to 150 mg, and not exceeding 750 mg mg per dose; wherein docosahexaenoic acid (DHA) is contained in an amount between 0.5 mg and 250 mg per dosage, preferably 1 mg to 100 mg, and more preferably 5 mg to 50 mg, and not exceeding 300 mg per dose.

### DETAILED DESCRIPTION OF THE PREFERRED EMBODIMENTS

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed considering the number of reported significant digits and by applying ordinary rounding techniques.

The present subject matter relates to compositions of matter and methods of preparation comprising non-cannabis derived, biologically diverse active agents, specifically including palmitoylethanolamide (PEA), the endogenous cannabinoid, docosahexaenoic acid (DHA), an omega 3 fatty acid, and beta-caryophyllene (BCP), the cannabinoid-like terpene. The compositions of the present subject matter may also include any and all additional plant components including but not limited to cannabinoids, (including, but not limited to, cannabichromene (CBC), cannabigerol (CBG), cannabidiol (CBD), and cannabidiolic acid (CBDA), n-3 fatty acids including, but not limited to, eicosapentaenoic acid (EPA), terpenes, including, but not limited to, humulene, myrcene, linalool, and pinene, carotenoids including, but not limited to, β-carotene, lycopene, astaxanthin and lutein and other herb and plant-based components including, but not limited to, wintergreen, capsaicin, and chlorophyllin, that employed over the mammalian lifespan, can modulate inflammatory diseases in order to mitigate age-related discomfort. Depending on application various herbs and flavonoids including, but not limited to, quercetin, luteolin, apigenin and vitexin, curcumin and green tea extracts, as well as certain excipients including, but not limited to, dimethyl sulfoxide, aloe vera and silicon dioxide may also be incorporated into the composition. These composition components are intended to positively affect the functioning of the mammalian endocannabinoid system (eCS)—and its regulation of homeostasis—subject to functional weakness due to loss of robust eCS tone, resulting in increased lifespan chronic inflammation response and therefore increased age-related discomfort.

At the center of age-related discomfort is pain, commonly resulting from tissue and/or nerve damage typically due to chronic immune system inflammation including, but not limited to low grade chronic inflammation. Chronic pain lasts or recurs for more than 3 to 6 months persisting past normal healing time. Chronic pain is a frequent condition calling for improved analgesics, affecting an estimated 20% of people worldwide. Pain perception depends on signaling to and from the brain involving a well-functioning, robust endoCannabinoid System (eCS). The eCS is the key to regulating homeostatic signaling (including down-regulation of pain signals) throughout the mammalian body and brain including immune system inflammatory response. The eCS can lose its robust tone due to stresses of life and aging resulting in greater incidence of excess or chronic inflammation, often due to inefficient resolution of inflammatory debris, over the lifespan and thereby contributing to chronic inflammation and further extending inflammatory tissue/nerve damage and accompanying pain or age-related discomforts.

Different naturally occurring Cannabinoids are found in plants (i.e., phytocannabinoids) and within mammals (i.e., endocannabinoids) but all are active neurotransmitters involving the mammalian eCS. These cannabinoids are known to interact synergistically in diverse entourage groups involving various neurotransmitter receptors, ion channels, etc., with a variety of similar compounds including, but not limited to, cannabinoids, terpenes, flavonoids, and terpenoids to produce a more powerful entourage effect than when individually acting alone. When functioning properly, natural endogenous cannabinoids (endocannabinoids), including but not limited to ANA, 2AG and PEA, are the basis for maintaining a functionally robust eCS tone in mammals.

When natural mammalian endocannabinoid production is inadequate for a variety of reasons (e.g., aging, stresses of living, certain illnesses, poor nutrition, chronic systemic inflammation, etc.), plant derived ‘phyto’ cannabinoids (e.g., cannabidiol or CBD) can stimulate the components comprising the eCS to rejuvenate their functionality in order to achieve a more robust activity level or a more robust eCS tone. Cannabis derived CBD (and THC) are legally problematic due to their continuing federally controlled substance history and status. In contrast, the present subject matter in some embodiments can be a cannabis-free composition to maintain a robust eCS tone across the patient's lifespan, thereby legally mitigating age-related discomfort.

### BCP (Beta-Caryophyllene)

BCP (beta-caryophyllene) is one of the major active essential oil components of various spice and food plants (e.g., pepper, basil, and cloves). Like CBD, BCP is a selective CB2 Cannabinoid receptor agonist that has analgesic attributes and, due to the entourage effect, their benefits are greater than the sum of their parts. Cannabinoid receptor agonists have shown therapeutic value against inflammatory and neuropathic pains, conditions that are often refractory to therapy. When activated, CB2 receptors—expressed by immune cells—can affect the release of chemical messengers (e.g., cytokines by immune cells) and can modulate immune cell trafficking. Activation of the CB2 receptor is a potential therapeutic strategy for the treatment of inflammation, pain, atherosclerosis, and osteoporosis, all involved in age-related discomfort.53 For example, BCP's biological entourage effects, acting through the same CB2 receptor that is responsive to CBD, include anti-inflammatory, anti-oxidative, and analgesic activities like that of CBD.5, 6, 34

Furthermore, the CB2 receptor activation has been shown to be devoid of psychotropic, adverse effects, which are frequently observed with CB1 receptor modulation. Among the cannabinoid ligands, β-caryophyllene (BCP) generated enormous therapeutic interest due to its noteworthy identification as a fully selective agonist of CB2 receptors, its affinity and binding with CB2, receptors along with favorable physicochemical and pharmacokinetic properties.53 It is one of the widely available dietary phytocannabinoids and is commonly used as a preservative, additive, and flavoring in food and cosmetics. It has been added to the list of “generally regarded as safe” compounds for dietary use by the United States Food and Drug Administration.53 Chemically, BCP is a bicyclic sesquiterpene abundantly found in the essential oils of different species such as Cinnamomum spp. and Piper spp.53 BCP is a secondary metabolite predominantly found in many dietary plants that exhibits potent and long-lasting antioxidant and anti-inflammatory properties in different models of human diseases. BCP has been shown to elicit potent pharmacological properties such as anti-inflammatory, antioxidant.53 Pharmacologically, it has been reported to be a CB2 receptor—selective agonist with a Ki value of 155 nmol/L for human CB2 receptors, with no affinity for CB1 receptors, which causes activation of Gi/Go subtype of G-proteins53. The identification and participation of CB2 receptors in mediating neuroprotection has driven special interest in the pharmacological investigation of BCP for neurodegenerative disorders including Parkinson's Disease.

### DHA (Docosahexaenoic Acid)

Cannabis plants, including HEMP, contain many components including seeds. Hemp seeds, for example, contain a significant amount of protective dietary n-3 polyunsaturated fatty acids (PUFA), particularly docosahexaenoic acid (DHA). This fact leads to the surprising conclusion that DHA has provided survival characteristics to the cannabis plant. A carrier component of many commercially available CBD oils, DHA is a widely available G.R.A.S. natural nutritional supplement from non-cannabis sources such as pure form in algae and in fish oil. DHA, a unique fatty acid that significantly alters basic properties of cell membranes, an important essential oil that must be supplemented, is a modulator of a mammalian host's inflammatory/immune responses. For example, DHA can attenuate pro-inflammatory cytokines for the treatment of autoimmune and chronic inflammatory diseases. But simply halting the inflammatory process is not enough to limit damage to tissue and nerves. There remains the issue of inflammatory debris resolution. Resolving inflammation is an active process for rebuilding tissue and removing the dead bacteria and cells. When that process is disrupted, inflammatory diseases arise.54

Advances in our understanding of the mechanisms that bring about the resolution of acute inflammation have uncovered a new genus of pro-resolving lipid mediators that include the lipoxin, resolvin, protectin and maresin families, collectively called specialized pro-resolving mediators. Research into the mechanism of chronic inflammation showed that Omega 3 PUFAs especially DHA was able to modulate the white blood cell macrophages to switch from pro-inflammatory cells to anti-inflammatory cells that enabled the process of resolving the debris resulting from inflammatory activity so that the process of healing and return to homeostasis could proceed.55 DHA can be metabolized into DHA-derived specialized pro-resolving mediators (SPMs). It is converted to 17-hydroperoxy-DHA derivatives via COX-2 and 15-LOX and 5-LOX activity. These derivatives are further converted into D-series resolvins and protectins with potent anti-inflammatory potential and potent neuroprotective effect.13 For these, and other, reasons, DHA is included with BCP and PEA in the present compositions.

### PEA (Palmitoylethanolamide)

The presence of PEA (and other structurally related N-acylethanolamines) has been known to enhance anandamide activity by an entourage effect whereby the complex system of receptors and ion channels responding to a variety of neurotransmitters, including but not limited to cannabinoids, terpenes, flavonoids, fatty acid amides, lipids, etc.61 PEA's analgesic and anti-inflammatory mode of action is by activating a nuclear receptor, the Peroxisome Proliferator-Activated Receptor alpha (PPAR-alpha), a master-switch for a great number of genes activating inflammatory cascades. In other words, PEA uniquely resets over-active genes, which code for inflammation.56 Therefore, PEA in conjunction with other terpenoid/cannabinoid compounds including but not limited to BCP and essential oils including, but not limited to DHA, and flavonoids including but not limited to quercetin and luteolin, described herein, can result in a boosting of eCS performance for a robust eCS tone in order to prophylactically minimize future age related discomforts. Just as there are a multitude of components (e.g., compounds, enzymes, ion channels, neurotransmitters, etc.), involved in the processing of pain, the contemporary use of the several primary active compounds described herein show an unexpectedly significant synergistic effect on hyperalgesia and neuropathic pain with respect to the single active ingredients acting alone.57

PEA is a fatty acid amide endocannabinoid discovered in 1957 in egg yolks that prevented rheumatic fever when fed to poor children with known streptococcal infections. Subsequently, PEA was found to alter the course of influenza.58 PEA is currently being studied as an adjuvant in treating COVID-19 infection.59 Palmitoylethanolamide (PEA) is synthesized by healthy tissue in the human body in response to inflammation.15 It works as a signaling molecule to down-regulate the inflammatory response of glial cells and mast cells.60 PEA is made by various plants and animals and is present throughout the animal kingdom. It can be extracted from natural sources, but modern production methods typically synthesize it from palmitic acid. PEA, available as a nutraceutical supplement, with a significant number of prospective and randomized trials demonstrating its pain-relieving effects with no reported drug-drug interactions and very few reported adverse effects from PEA.58 PEA has been studied in in vitro and in vivo systems using exogenously dosed compound with evidence that it binds to the peroxisome proliferator-activated receptor alpha (PPAR-α) 16 through which it exerts a variety of biological effects, some related to mitigation of chronic inflammation and pain.17, 61

In addition to the peroxisome proliferator-activated receptor alpha (PPAR-α)16, 17, 62, PEA, unlike ANA or 2AG, also has affinity to cannabinoid-like G-coupled receptors GPR55 and GPR119.33 While PEA lacks complete affinity for the CB1 and CB2 cannabinoid receptors,63 it is a full agonist of the GPR55 receptor now considered to be the CB3 receptor.64, 65 Primary research supports that the presence of PEA enhances anandamide activity by an entourage effect66, 67 and that PEA levels are altered and that the endocannabinoid system (ECS) is “imbalanced” in acute and chronic inflammation.68 PEA has been shown to have anti-inflammatory,69 anti-nociceptive,70 neuroprotective,71 and anticonvulsant properties.72

PEA is a nutraceutical endocannabinoid that was retrospectively discovered in egg yolks. Feeding poor NYC school children with known streptococcal infections prevented rheumatic fever. Subsequently, it was found to alter the course of influenza. Since 2008, PEA, available from various foods (e.g., egg yolks, peanuts etc.,), has been available as a nutraceutical under the various brand names. A literature search on PEA has yielded over 350 papers, all referenced in PubMed, describing the physiologic properties of this endogenous modulator and its pharmacologic and therapeutic profile as an anti-inflammatory endocannabinoid.73 Unlike CBD, Palmitoylethanolamide targets nonclassical CB3 cannabinoid receptors rather than CB1 and CB2 receptors. Palmitoylethanolamide will only indirectly activate classical cannabinoid receptors by an entourage effect. There are a considerable number of prospective and randomized trials demonstrating the pain-relieving effects of PEA, including micronized and ultra micronized forms.74

There are no reported drug-drug interactions and very few reported adverse effects from PEA.58 The endocannabinoid PEA, a fatty acid amide discovered in 1957, has been studied in in vitro and in vivo systems using exogenously dosed compound with evidence that it binds to a nuclear receptor, the peroxisome proliferator-activated receptor alpha (PPAR-α)16 through which it exerts a variety of biological effects, some related to chronic inflammation and pain.17, 61

PEA, a naturally occurring endocannabinoid not found in cannabis, is associated with major effects in mitigating neuroinflammatory processes particularly in the brain.9, 75 It is generally known that neuroinflammation has an important role in the causation and maintenance of chronic pain. This process is characterized by infiltration of immune cells, activation including granulation of mast cells and glial cells (as glial cells are emerging as a major factor in chronic pain), resulting in production of inflammatory mediators in the peripheral and central nervous system involving the eCS.17, 58 PEA is an anti-inflammatory and pro-resolving lipid mediator which controls glial and mast cell behaviors associated with pain. PEA down-modulates immune system mast cell activation response to tissue injury releasing inflammatory cytokines, NGF, histamines, and other molecules that attract white blood cells, thereby activating their nociceptor pathogen immune response.76, 77

PEA induced pain relief is progressive, age- and gender independent. As an acylethanolamine, PEA readily disperses throughout different tissues in the body and brain, including nervous tissues.17 Similar to ANA and 2AG, PEA is synthesized on demand when its endogenous levels, systemic or local, are altered. This occurs in circumstances of stress, aging, injury and/or pain. PEA, as a natural endocannabinoid, has no known acute or chronic toxicity nor significant side effects.58

PEA has a high efficacy/risk ratio, has no known tolerance effects, and does not interfere with concurrent therapies for pain or for co-morbid conditions. The effects of PEA are dose-dependent; long-term treatment with PEA has been shown to not only reduce pain but actually to preserve peripheral nerve function and mitigate brain inflammation.58 Supplemental PEA, as a natural endocannabinoid, has capabilities to increase the robustness of the eCS as other cannabinoids (both endo- and phyto-) and therefore contribute prophylactically to reduction of age related discomfort. The effects of ANA and 2-AG can be that it works via adapting the cellular metabolism.78 This most probably is one of the main reasons for its analgesic and anti-inflammatory activity.79, 80

### Astaxanthin

Astaxanthin (ASX) is a uniquely powerful component of our composition of matter that, in addition to adding additional anti-inflammatory effects of its own, is able to provide surprisingly powerful protection of our lipids from fierce inflammatory oxidation.81 A lipid-soluble carotenoid with a unique molecular structure, ASX is responsible for powerful antioxidant activities by simultaneously quenching singlet oxygen and scavenging free radicals.82 Synthesized naturally by many types of marine life ASX primarily enhances the action of PPARα and suppresses that of PPARβ/δ and PPARγ, but also displays the opposite effects on PPARs, depending on the cell context.83 Anti-inflammatory effects of ASX are mediated by PPARγ activation able to diminished the gene expression of inflammatory cytokine activity in macrophages.84 The US FDA allowed the use of ASX as a dietary supplement in 1999.85

### Neuroinflammation

Modern neurosciences consider neuroinflammation as “any inflammatory process, both acute and chronic, that affects the central or peripheral nervous system.”86,87 Neuroinflammation is a physiological response aimed at maintaining the homodynamic balance and providing the body with the fundamental resource of adaptation to endogenous and exogenous stimuli. Although the response is initiated with protective purposes, the effect may be detrimental when not regulated.88 The physiological control of neuroinflammation is mainly achieved via regulatory mechanisms performed by particular cells of the immune system intimately associated with or within the nervous system and named “non-neuronal cells.”89 In particular, mast cells within the central nervous system and in the periphery90 and microglia (at spinal and supraspinal level) are involved in this control, through a close functional relationship between them and neurons (either centrally, spinal, or peripherally located).91 Accordingly, neuroinflammation becomes a worsening factor in many disorders whenever the non-neuronal cell supervision is inadequate. It has been shown that the regulation of non-neuronal cells—and therefore the control of neuroinflammation—depends on the local “on demand” synthesis of the endogenous lipid amide Palmitoylethanolamide and related endocannabinoids.78 When the balance between synthesis and degradation of this bioactive lipid mediator is disrupted in favor of reduced synthesis and/or increased degradation, the behavior of non-neuronal cells may not be appropriately regulated and neuroinflammation exceeds the physiological boundaries. Hyper-activated non-neuronal cells like mast cells and microglia92, 93 as well as astrocytes (i.e., Neuroglial cells that do not belong to the immune system94 are importantly involved in neuroinflammatory processes. mutually interact with each other through a cytokine-mediated cross-talk that can amplify or chronicize neuronal suffering.95 If uncontrolled, non-neuronal cells are thus considered to play an important role in the induction and maintenance of peripheral and central sensitization, associated with inflammatory pain, chronic and neuropathic pain, and with many clinical conditions of dysmetabolic, traumatic, or degenerative nature.91, 97, 98

In these conditions, it has been demonstrated that the increase of endogenous Palmitoylethanolamide—either by decreasing its degradation or exogenous administration—is able to keep neuroinflammation within its physiological limits.99, 100, 101

### Entourage Compounds

The present subject matter relates to a unique composition enhanced by “entourage compounds” such as N-palmitoylethanolamide (PEA) that inhibit their hydrolysis via substrate competition, and thereby prolong their action.9

A main target of PEA is the peroxisome proliferator-activated receptor alpha (PPAR-α).16, 62 PEA also has affinity to cannabinoid-like G-coupled receptors GPR55 (recognized as cannabinoid receptor CB3) and GPR119.33 While PEA lacks complete affinity for the CB1 and CB2 cannabinoid receptors,63 primary research supports that the presence of PEA enhances anandamide activity by an entourage effect.57, 66, 67 Primary research supports the conclusion that PEA levels are altered and that the endocannabinoid system (eCS) is “imbalanced” in acute and chronic inflammation.68 PEA has been shown to have antiinflammatory,69 anti-nociceptive,70 neuroprotective,71 and anticonvulsant properties.72 PEA has an interesting mode of action, it actives a nuclear receptor, the Peroxisome Proliferator-Activated Receptor alpha (PPAR-alpha), which is a master-switch for a great number of genes activating inflammatory cascades PEA resets over-active genes, which code for inflammation, and in order to reset these via the PPAR receptor.102 This composition of matter including BCP, DHA and a naturally occurring PEA—a CB3 receptor agonist and considered a ‘super-cannabinoid’, PEA nutritional supplement available only from natural non-cannabis sources.103 PEA is able to perform the function of maintaining a robust eCS tone in a similar way to that of CBD. PEA is endogenously produced on-demand in all tissue cells, as a protective response to injury, stress, inflammation, and pain.104, 105, 106

When pain is protracted, however, PEA ‘exhaustion’ may develop. Chronic inflammatory conditions create lower levels of PEA. The exogenous administration of PEA may in such cases serve to replenish levels of endogenous PEA, restoring its protective, anti-inflammatory, and analgesic effects.105, 106

Maintenance of a robust endocannabinoid system (eCS) functionality or ‘tone’ across a mammal's lifespan is supported by cannabinoid supplementation.7 Legal and regulatory issues make use of the cannabinoid entourage effect from Cannabis problematic.107 The present subject matter eliminates the issues surrounding Cannabis by completely avoiding any involvement of Cannabis derived compounds. This subject matter is based on a little-known non-cannabis derived cannabinoid, Palmitoylethanolamide (PEA).108

PEA, in combination with two other natural generally recognized as safe (GRAS) compounds, Beta-Caryophyllene (BCP) and DocosaHexaenoic Acid (DHA), and at least one other anti-inflammatory compound, describes a unique composition of matter.109 The composition mimics the eCS entourage effect of cannabis in support of a robust eCS tone necessary for effective, robust regulation of homeostasis for anti-inflammatory balance without relying on CBD (Cannabidiol) or any other Cannabis derived compounds such as THC.76 The objective is to maintain a robust eCS tone necessary to properly regulate homeostasis in order to keep inflammation in balance, thereby minimizing chronic inflammation; over the lifespan and therefor reduce potential for age-related discomfort in living mammals.110, 111

The present subject matter relates to compositions and methods of preparation comprising the non-cannabis derived biologically diverse active agents palmitoylethanolamide (PEA), an endogenous cannabinoid, docosahexaenoic acid (DHA), an omega 3 fatty acid, beta-caryophyllene (BCP), a cannabinoid-like terpene, and any plant components including but not limited to cannabinoids, terpenes, flavonoids, and terpenoids, including but not limited to cannabichromene (CBC), cannabigerol (CBG), cannabidiol (CBD), cannabidiolic acid (CBDA), humulene, myrcene, linalool, quercetin, and pinene, intended to positively affect the functioning of all mammalian endocannabinoid systems subject to functional weakness resulting in age related discomfort.1, 9 At the center of age related discomfort is pain, typically resulting from tissue and/or nerve damage due to chronic immune system inflammatory response.42 Chronic pain lasts or recurs for more than 3 to 6 months112 persisting past normal healing time.113 Chronic pain is a frequent condition calling for improved analgesics, affecting an estimated 20% of people worldwide.2, 3, 4 Pain perception depends on signaling to and from the brain and a well-functioning endocannabinoid system (eCS), the key to regulating homeostatic signaling throughout the mammalian body and brain.114

These cannabinoids interact synergistically in a diverse entourage of groups with a variety of similar compounds including, but not limited to, terpenes to produce greater ‘entourage’ effects than when individually acting alone. Naturally occurring Cannabinoids are active through the eCS in all living mammals. The effects of ANA and 2-AG can be enhanced by “entourage compounds” that inhibit their hydrolysis via substrate competition, and thereby prolong their action. Entourage compounds includes N-palmitoylethanolamide (PEA).9

When functioning properly, natural endogenous cannabinoids (endoCannabinoids), including but not limited to ANA, 2AG and PEA, are the basis for maintaining a functionally robust eCS tone.115 When natural mammalian endocannabinoid production is inadequate for a variety of reasons (e.g., aging, stresses of living, certain illnesses, poor nutrition, lack of physical activity, etc.), plant derived ‘phyto’ cannabinoids (e.g., Cannabidiol or CBD) can stimulate the components comprising the eCS to increase their number and rejuvenate their functionality and general availability in order to achieve a more robust activity level or a more robust ‘CS tone’.1 Beta-Caryophyllene (BCP), a terpene produced in a wide variety of non-cannabis plant sources, is a Cannabinoid receptor agonist that interacts with various receptors, including but not limited to, CB2, the same way as CBD and BCP as anti-inflammatory.116 When activated, CB2 receptors—expressed by immune cells—can affect the release of chemical messengers (e.g., cytokines by immune cells) and can modulate immune cell trafficking. Activation of the CB2 receptor is a potential therapeutic strategy for the treatment of inflammation, pain, atherosclerosis, and osteoporosis.5, 60

The presence of PEA (and other structurally related N-acylethanolamines) has been known to enhance anandamide activity by an entourage effect whereby the complex system of receptors and ion channels responding to a variety of proximal neurotransmitters, including but not limited to cannabinoids, terpenes, flavonoids, fatty acid amides, lipids, etc.114, 117 Therefore, PEA in conjunction with other terpenoid/cannabinoid compounds including but not limited to BCP described herein can result in a boosting of eCS performance for a robust eCS tone in order to prophylactically minimize future age related discomforts through, for example, reduction of chronic inflammation.77 Just as there are a multitude of components (e.g., compounds, enzymes, ion channels, neurotransmitters, etc.), involved in the processing of pain, the contemporary use of the several primary active compounds described herein show an unexpectedly significant synergistic effect on hyperalgesia and neuropathic pain with respect to any single active ingredient acting alone.57, 96

### Inflammation & Natural Herbs

The incidence of diseases with inflammatory etiopathology have recently increased. Drugs relieving these ailments also produce serious life-threatening consequences.118 Research on medicinal herbs and the endocannabinoid system have opened a new era in the prophylactic and therapeutic management of inflammatory diseases. Medicinal plants or their constituents are considered beneficial due to satisfactory potency, ease of availability, cost effectiveness, much less or no side effects, safer and efficient as compared to synthetics. Natural products including medicinal herbs containing phytoconstituents can prevent undesirable inflammatory processes and possess anti-inflammatory activity.30 Inflammation is part of the complex biological response of body tissues to harmful pathogens, damaged cells, or irritants, and is a protective response involving immune cells, blood vessels, and molecular mediators. The inflammatory response is designed to eliminate the initial cause of cell injury to help fight and clear necrotic cells, and repair damaged tissue and organ systems. Too little inflammation can lead to progressive tissue destruction by the harmful stimulus (e.g., bacteria) potentially compromising organism survival. Although this process is protective, the failure to resolve the inflammation and/or recognize the return of the target tissue to homeostasis can result in chronic systemic inflammation.54 Chronic inflammation is associated with various autoimmune and chronic inflammatory diseases such as rheumatoid arthritis, inflammatory bowel disease atherosclerosis, including the promotion of cancer.119

### Resolvins & Protectins

The resolution of inflammation is an active process controlled by endogenous mediators with selective actions on neutrophils and monocytes. The initial phase of the acute inflammatory response is characterized by the production of pro-inflammatory mediators followed by a second phase in which lipid mediators with pro-resolution activities may be generated. The identification of these mediators has provided evidence for the dynamic regulation of the resolution of inflammation. Among these endogenous local mediators of resolution, lipoxins (LXs), lipid mediators typically formed during cell-cell interaction, were the first to be recognized. More recently, families of endogenous chemical mediators, termed resolvins and protectins, were discovered. LXs and aspirin-triggered LXs are considered to function as ‘braking signals’ in inflammation, limiting the trafficking of leukocytes to the inflammatory site.

LXs are actively involved in the resolution of inflammation stimulatingnon-phlogistic phagocytosis of apoptotic cells by macrophages. Furthermore, LXs have emerged as potential anti-fibrotic mediators that may influence pro-fibrotic cytokines and matrix-associated gene expression in response to growth factors.10 DHA, a key ingredient of our composition of matter, gives rise to resolvins and related compounds (e.g., protectins) through pathways involving cyclooxygenase and lipoxygenase enzymes to resolve the inflammatory responses.13

### B Complex Vitamins

In other embodiments, the present methods and compositions can further include one or more B vitamins, or B-complex vitamins. The inclusion of such B vitamins may be of assistance in treating chronic inflammation and age-related discomforts generally.

Since the late 1980s, the role of B vitamins on analgesia have been a topic of research.120 All B vitamins have important roles as coenzymes and precursors for enzymatic reactions in different biological systems.121 While these roles are different, they are related, and all B vitamins as discussed herein can be considered individually or grouped together as “B-complex” vitamins.122 Suitable, non-limiting examples of such B vitamins include thiamine (vitamin B1), pyridoxine (vitamin B6), and cyanocobalamin (vitamin B12). It is postulated that these vitamins, given at therapeutic doses (higher than physiologic replacement doses), alleviate pain when given alone, in combination, and as adjunct therapy alongside NSAIDs and other non-opiate analgesics. It is contemplated that when used in the present compositions, any of the B vitamins mentioned may have clinical roles including, but not limited to, the reduction of neuropathic pain, burning, and itching, inflammation, lower back pain, and postoperative discomfort. For example, these vitamins can potentially improve pain scores in conditions such as carpal tunnel, migraine, fibromyalgia, and premenstrual tension.123

Such benefit from B-vitamin supplementation may result from the correction of underlying deficiencies, as well as direct analgesic and antinociceptive action throughout the body, and especially in the peripheral nervous system.124 B complex vitamins not only contribute to important physiological functions as co-factors, antioxidants, and structural components in the whole human body, but they also possess neurospecific functions.125 More recently, these vitamins have been found to, at times, inhibit the nociceptive activity in neurons found in the dorsal horn of the spinal cord and in the thalamus.126 As evidenced by decreased C-reactive protein serum levels found in patients with B-complex supplementation as well as B12 monotherapy, these vitamins could play a direct role in systemic inflammation.127

B vitamins are not made in the mammalian body and must be ingested. Even with a well-balanced diet, Thiamine (B1) can be obtained from dairy, whole grain, and red meat and recommended dietary dosage is about 1.2 mg/day, although therapeutic doses of up to 750 mg/day have been studied and are regarded as safe.122 Pyridoxine (B6) sources include fish, beef liver, starchy vegetables, and non-citrus fruits. Recommended daily dosage of pyridoxine range from about 0.3 mg per day in infants, to about 1.7 mg per day in adults.128 While B vitamins are water soluble and easily excreted, extremely high doses of pyridoxine over time are associated with various acute and chronic neuropathies. Pharmacologic doses of pyridoxine shown to provide analgesic benefits range between about 5 and about 50 mg per day. Cyanocobalamin (vitamin B12) and its “activated” form, methylcobalamin (methyl-B12), are popular supplements and well known for their clinical roles in neuropathic pain. Dietary sources of vitamin B12 include fish, meat, poultry, eggs, and dairy. Recommended daily intake is between about 0.5 and about 2.4 mcg/day for children and adults, respectively, while therapeutic doses ranging from about 0.3 mg to about 10 mg per day may be safe and effective in various pain models and as adjunct therapy for inflammatory pain.122

### Herbs and Phyto-Supplements

### DHA (Docosahexaenoic Acid)

Much published literature supports the contention that dietary n-3 polyunsaturated fatty acids (PUFA), docosahexaenoic acid (DHA) in particular, is an important modulator of a host's inflammatory/immune responses and for the treatment of autoimmune and chronic inflammatory diseases.129 DHA (docosahexaenoic acid) is a unique fatty acid, because it significantly alters basic properties of cell membranes, including fatty acid chain order and fluidity, phase behavior, elastic compressibility, ion permeability, fusion, rapid flip-flop, and resident protein function.129 Another key finding is that DHA is able to reverse the macrophages pro inflammatory action to one of anti-Inflammatory resolving action.54 DHA gives rise to resolvins and related compounds (e.g., protectins) through pathways involving cyclooxygenase and lipoxygenase enzymes to resolve the inflammatory responses.13, 130

### BCP (Beta-Caryophyllene)

Chemically, beta-caryophyllene (BCP) is a bicyclic sesquiterpene that exhibits potent and long-lasting anti-inflammatory and antioxidant properties in different models of human diseases53 beta-caryophyllene (BCP), acting as a phytocannabinoid, is sourced from various non-cannabis sources is ‘Generally Recognized as Safe’ (GRAS) and FDA approved for food use.53

BCP selectively binds to the CB2 receptor, acting as a strong CB2-selective agonist. CB2 activation can mediate anti-nociception either directly or indirectly by inhibiting IL-1beta and TNF-α; TRP-1, TRP-2, NO, IL-1beta, IL-6, IL-8, IL-12. IL-17, where direct activity is exerted through CB2 stimulation on primary sensory neurons.131 BCP displays similar analgesic activities as several essential oils, in which BCP is a major active compound (e.g., basil and rosemary). One can hypothesize that better analgesic effects may be obtained when BCP is used in combination with other natural agents of desired properties. In inflammatory hyperalgesia, indirect pain inhibition through CB2 localized on mast and immune cells is achieved by the reduction of prostanoids or cytokines release, which are responsible for peripheral nociceptor sensitization that tissue damage leading to a painful feeling, is occurring. BCP inhibits proinflammatory cytokine expression in peripheral blood. One can hypothesize that better analgesic effects may be obtained when BCP is used in combination with other natural agent(s) of desired properties. BCP has the potential therapeutic efficacy to elicit significant neuroprotection by its anti-inflammatory and antioxidant activities mediated by activation of the CB2 receptors.132 BCP may act in a comparable manner to other CB2-selective agonists. CB2 activation can mediate anti-nociception either directly or indirectly, where direct activity is exerted through CB2 stimulation on primary sensory neurons. In inflammatory hyperalgesia, indirect pain inhibition through CB2 localized on mast and immune cells is achieved by the reduction of prostanoids or cytokines release, which are responsible for peripheral nociceptor sensitization.6

### Cannabinoids and the Endocannabinoid System

The eCS homeostatic control mechanism is composed of endocannabinoids, which are endogenous lipid-based retrograde neurotransmitters that bind to or impact various cannabinoid receptors, and cannabinoid receptor enzymes expressed throughout the vertebrate central nervous system including the brain and peripheral nervous system. The “classic” endocannabinoid system (eCS) includes the primary cannabinoid receptors CB1 and CB2, the eCB neurotransmitter ligands anandamide (ANA), 2-arachidonoylglycerol (2-AG) and palmitoylethanolamide (PEA) and their metabolic enzymes including fatty acid amide hydrolase (FAAH) or monoacylglycerol lipase (MAGL), responsible for just-in-time synthesis and degradation of the receptors needed in maintaining homeostasis for proper health and wellness. Wellness is when physiological processes and systems are functioning properly so that everything is ‘in balance.’ Homeostasis is the process of maintaining this complex system-wide balancing act near physiological balance necessary for lifespan health and wellness. Inflammation is a ‘far from homeostasis’ condition designed to repair minor imbalances while chronic inflammation is a health disruptor.115 The CECD syndrome can occur when the eCS loses its robust tone and is unable to maintain homeostasis thereby allowing inflammation to support various disorders, illnesses and conditions potentially contributing to various additional age-related discomforts.24

A systematic review of clinical interventions that enhance the operational performance of the eCS, (i.e., ways to up-regulate cannabinoid receptors, increase ligand synthesis, or inhibit ligand degradation) identifies the phytocannabinoid cannabidiol (CBD)—an analog of the endocannabinoid 2AG—as an efficacious natural compound of cannabis plants able to maintain a robust eCS tone (a measure of eCS ability to do its job efficiently.) Currently, however, CBD along with all other cannabis derived phytochemical compounds are guilty by association with tetrahydrocannabinol (THC), the only component of cannabis (i.e., marijuana and hemp plant families) with potentially harmful psychological effects including intoxication and potential brain damage with continuous and/or elevated levels of ingestion and/or inhalation.4

The drug Epidiolex (containing 95% CBD) has been approved by the FDA to treat pediatric seizures (i.e., Lennox-Gastaut and Dravet syndromes).42 Because CBD is now a drug, FDA is enabled to further limit the casual use of CBD for treating any health condition without FDA approval, requiring a costly and time consuming gold standard clinical trial process for each indication.42 Alternative cannabis-derived cannabinoids are available to potentially maintain eCS tone. However, these compounds are also associated with the legal restrictions on cannabis and so are currently impractical as candidates for classification as ‘generally recognized as safe’ (GRAS). The present subject matter describes a novel and unexpected combination of at least some non-Cannabis derived cannabinoids, terpenes, fatty acids and other nutritional supplements in combination able to safely maintain a robust eCS tone promoting regulation of inflammation and reduced pain signaling to prevent at best and mitigate at worst Age Related Discomfort.

The literature describes common over the counter compounds including various typically used Non-Steroidal Anti-Inflammatory Drugs (NSAIDs) (e.g., aspirin, Advil, Celebrex, ibuprofen (Motrin), naproxen (Aleve, Naprosyn), etc.) that relieve pain and reduce inflammation. However, over the counter NSAIDS, although legal, cause recently described long term effects (e.g., gastric ulcers) in addition to common short-term nausea, vomiting, diarrhea, constipation, rash, headache and bleeding. Cannabinoids are scientifically recognized as natural anti-inflammatory compounds with superior efficacy versus NSAIDs without their long term use effects which includes risk of gastric ulcers and potential increased risk of coronary heart disease.19, 20 Furthermore, NSAIDs do not maintain the critical robust eCS tone condition that cannabinoids do. Cannabinoids interact with the eCS to reduce inflammation and down-regulate related pain without serious side effects. Until CBD and other cannabis derived compounds are deregulated or made federally legal to use by the FDA, FTC, and DEA, they are problematic as nutritional supplements.133 In future, the present compositions may further include Cannabis derived compounds when they are no longer listed as Class 1 controlled substances.133

The science of the endocannabinoid system is relatively new and not yet taught in medical/veterinary schools. In addition, the pharmacology of cannabis plants is not well understood because of its prohibition as a class one controlled substance which tightly restricted plant material for US research. As such, the more than 100 phytocannabinoids and over 200 terpenes included in cannabis plants are still not well understood.69 A limited number of terpenes from a variety of non-cannabis plants have been used primarily as fragrances in cosmetics as well as in aroma therapy. They are not discussed in relation to the eCS. DHA, commonly known as fish oil, is recommended by doctors for maintaining cardiovascular health, especially controlling triglycerides implicated in arteriosclerosis.

The present subject matter describes non-obvious combinations of certain natural non-cannabis derived compounds (and their methods of preparation) including but not limited to N-acylethanolamines/cannabinoids (e.g., palmitoylethanolamide or PEA) possessing significant analgesic properties through eCS regulated homeostasis including, but not limited to, down regulation of nociception and pain which is common to age-related discomfort in humans and companion animals. Over the mammalian lifespan, maintenance of a more robust eCS tone will reduce unnecessary chronic inflammatory response. Maintenance of a robust eCS tone will result in reduced inflammatory disease implicated in chronic tissue damage, for example, and therefore reduced age-related discomfort.

The repeated frustration of not being able to use CBD from Hemp oil in companion animals and humans in competitive sports resulted in a search for a new composition of matter that acted on the eCS like Hemp oil (with its hundreds of components) acting together in a coordinated entourage effect on the process of homeostasis regulation.114 After serious effort to identify the various receptors involved in pain, the operation of the eCS and then researching the various components of cannabis to find a terpene that was able to act like CBD—but not be from cannabis—the molecule known as BCP was identified. Finally, a third primary component with influence in the inflammatory process was inferred from the chemistry of hemp seed oil rich in DHA, an endocannabinoid precursor. There is nothing obvious in the literature that connected these three components acting together especially not involving the eCS or inflammation.60, 134

A surprisingly robust eCS tone effect is obtained by administering several anti-inflammatory and or antioxidant compounds in combination with PEA (Palmitoylethanolamide) including DHA (Docosahexaenoic acid) and BCP (Beta-caryophyllene) at the same time, at particular dosages and in combination with various analgesic/anti-inflammatory ingredient/excipients examples of which are listed in Table 1. Stated another way, the contemporary use of the three active compounds (i.e., PEA, DHA and BCP) in specific combination shows an unexpected synergistic effect on hyperalgesia and neuropathic pain with respect to the single active ingredients administered alone.129, 115 Depending on formulation and modes of administration in mammals, the present subject matter can systemically enhance eCS tone which may reduce inflammatory pain through 1. the eCS and 2. other pain associated cell receptors and pathways including, but not limited to, CB1, CB2, GPR55, TRPV½, PPAR-α activation as well as, mast cells and glial cells throughout the mammalian brain and body.17, 73, 135

In addition to conventional eCS receptors, other associated cell receptors including but not limited to inhibitors, receptor signaling modulators (e.g., aminos acids, lipids, peptides/proteins, etc.) as well as others and non-receptor modulators (e.g., glial and mast cells), including but not limited to, ion channels and transporters located throughout mammalian brain/body physiology believed to impact issues such as pain associated with age related discomfort.1, 43, 68

Additional additives, including but not limited to, polyunsaturated fatty acids (PUFAs) such as Omega 3 DHA (Docosahexaenoic Acid), may be employed in enhancing the action of said anti-inflammatory and/or antioxidant compounds that would make it more effective in robust eCS tone maintenance.136

Certain additional plant derived compounds with anti-inflammatory and antioxidant properties, including but not limited to Chlorophyllin, a water-soluble liquid form of chlorophyll, will be useful in certain applications to make said composition more effective in eCS tone maintenance. Evidence has shown that chlorophyll has anti-inflammatory properties that may benefit those affected with chronic inflammation. In the journal Inflammation, researchers discovered that chlorophyll helped inhibit TNF-α (tumor necrosis factor-alpha) in mice. Chlorophyll supplements are generally considered safe in low doses, according to a 2014 study published in the Journal of Dietary Supplements.137

Various additives, including but not limited to, plant derived terpenes/terpenoids (e.g., beta caryophyllene (BCP)), that activate CB2 receptors may be employed in enhancing the performance of said anti-inflammatory and/or antioxidant compounds (i.e., PEA and DHA) to increase effectiveness of a robust eCS tone in addressing age related discomfort.16, 131, 138

Natural plant flavonoids including, but not limited to, certain phenolic amides (e.g., capsaicin), may be useful in certain applications in reducing pain associated with age related discomfort.139

Additional processing techniques including but not limited to ultrasonic cavitation in the generation of normally insoluble anti-inflammatory substances including, but not limited to, curcumin-loaded micelles. Such micelles could be employed to increase product aqueous solubility in addition to uniformity and efficacy which enables greater effectiveness in dealing with issues involved in age related discomfort.140, 141 Processing supplemental food products containing flavonoids such as quercetin, carotenoids such as astaxanthin, or polyunsaturated omega-3 fatty acid (DHA) using freeze-drying can allow for a long shelf life without degradation of beneficial actives, allowing for long storage times and almost no risk of microbial contamination. Freeze drying uses temperatures than traditional dehydration, which is beneficial for such ingredients.

Various additives including, but not limited to, absorbent excipients such as silicon dioxide (e.g., colloidal silicon dioxide), can enable essential oils and aqueous solutions to be transformed into solids including but not limited to powders or rapid dissolving tablets for oral ingestion by mammals to facilitate bioavailability. Such excipients can also facilitate various dosage form manufacturing processes.142

In addition, the present compositions can be administered as a daily dosage form that is an oral or topical dosage form. Non-limiting examples of topical dosage forms useful herein include liquids, creams, gels, ointments, foam, solutions, suspensions, lotions, and the like. Non-limiting examples of oral dosage forms useful herein include tables, capsules, liquids, and the like.

Certain solvents including, but not limited to, dimethyl sulfoxide (DMSO) capable of dissolving both polar and non-polar compounds is known to easily penetrate the skin of mammals including but not limited to humans and companion animals and to transport small molecules through biological membranes.143, 144 DMSO has also shown efficacy in inflammatory conditions including, but not limited to, rheumatoid arthritis, in modulating the production of pro-inflammatory cytokines/chemokines from human monocytes, the cell in human blood secreting these inflammatory mediators.113, 145

Certain antioxidants including, but not limited to, N-Acetyl Cysteine (NAC), PQQ, Astaxanthin and Glutathione. NAC, a precursor to glutathione, acts as an excipient free-radical scavenger in stabilizing unsaturated lipids such as DHA. NAC, by modulating the immune response and increasing natural glutathione levels, can further reduce inflammation. Clinical research and practice show NAC is effective, safe, and mostly well tolerated. Therapeutic activity of NAC relies on reduction of oxidative stress, modulation of mitochondrial dysfunction, apoptosis, inflammatory processes, and modulation of glutamate homeostasis.146 PQQ (Pyrroloquinoline Quinone) is a natural biofactor anti-oxidant small molecule that boost mitochondrial functioning and influences oxidative stress.147 Astaxanthin, a natural carotenoid and powerful antioxidant that reduces oxidative stress, is sourced from algae.148 Glutathione is a natural antioxidant peptide addressing oxidative stress.149

TABLE 1
EXAMPLES
EXAMPLE NON-CANNABIS INGREDIENT LIST FOR A COMPOSITION TO SUPPLEMENT
ENDOCANNABINOID SYSTEM TONE ADDRESSING AGE RELATED DISCOMFORT

| | | | | MECHANISM |
|---|---|---|---|---|
| INGREDIENT | DAILY DOSE | | | (Including ‘entourage’ |
| (eCSEPTIONOL ™) | RANGE | | FUNCTION | effect) |
| Topical or Supplement | Dosage Depends on Form | | Systemic Pain Management | Mechanism of Action |
| PEA | 25-3600 | mg | Inflammation | CB3 (PPAR-a, TRPV1, |
| | | | Management | NAPE-PLD, CB₁, CB₂) |
| beta-Caryophyllene | 10-750 | mg | Pain Signaling | CB₂ |
| Omega 3 DHA | 0.5-300 | mg | Inflammation Resolution | Activates PPAR-γ, TNFα |
| | | | | Resolvins/protectins |
| Astaxanthin ASX | 0.02 mg-30 mg | | Anti-oxidant effect and | Lipid Antioxidant, |
| | | | inflammation reduction | decreasing the mRNA |
| | | | | and protein expressions |
| | | | | of iNOS and COX-2 |
| Quercetin | 20-1400 | mg | Anti-oxidant effect and | Activates PPAR-γ |
| | | | inflammation reduction | activity, reduces |
| | | | | transcription of |
| | | | | inflammatory genes |

TABLE 2
Comparison of PEA vs CBD in mitigating inflammation & age-related discomfort

| Palmitoylethanolamide (PEA) | Cannabidiol (CBD) |
|---|---|
| Endogenous-natural made by the mammalian body | Not made in the mammalian body and must be |
| | extracted from cannabis |
| Natural Sources: egg yolk, peanuts, lecithin, | Natural Sources: cannabis/hemp and marijuana |
| sunflower oil, etc. | only |
| Never contains THC | Naturally includes traces—or more—of THC |
| No legal considerations hindering use. PEA is | Illegal in some States; Illegal in interstate |
| Generally Recognized As Safe by FDA and WHO | commerce; FDA approved as a drug. |
| Endogenous level decreases with age; data supports | Not endogenous to mammals. |
| benefits of supplementation | |
| Produced during eCS regulation of homeostasis in | Is not produced in the mammalian body at all |
| response to pain, stress, and tissue damage | |
| Activates PPAR-alpha receptor controlling | Genetic action not established; not shown to |
| numerous key genes in the pain process | activate PPAR receptors |
| Full Agonist of reputed CB3 receptor GPR55 | Full Antagonist of the GPR55 receptor |
| Hundreds of randomized clinical trials since 1970s | Until recently US clinical trials restricted due to |
| | US DEA Class 1 Controlled substance designation |
| No deleterious effects noted anywhere in the world | May have deleterious effects on fetus of pregnant |
| since mid-1970's | woman and may interfere with some Rx drugs |
| PEA is neuroprotective; greatly reduces | CBD is less abundant and less efficacious in the |
| inflammation in the brain post TBI trauma and | brain compared to PEA |
| stroke | |
| PEA both reduces inflammation as well as aids in | Simply down-regulates pain signaling of damaged |
| tissue healing | tissues; no reported aid to healing |
| High supplemental PEA inhibits eCS degrading | CBD not known to greatly inhibit enzymes that |
| enzymes; thereby extending effective pain reduction | degrade cannabinoids |
| Supplemental PEA enhances ANA availability, | Supplemental CBD does not significantly enhance |
| enhancing pain reduction | ANA availability |
| No drug-drug interactions reported; PEA is a pain | Interacts in liver with cytochrome P450 enzymes; |
| adjuvant; eventually replacing drugs | |
| Absolutely no side effects reported | Potential allergic side effects noted; FDA warns of |
| | use by pregnant women |
| PEA is not subject to farming/processing | CBD is plant derived; not federally regulated; can |
| contamination (e. g., heavy metals, bacteria) | contain THC & dangerous heavy metals |
| PEA is therapeutic in Parkinson's Disease (PD) trials; | No reports of CBD in PD clinical trials. CBD not |
| PEA is neuroprotective; PEA is prophylactic in TBI | as active or neuroprotective in the brain as PEA |

Any embodiment of the present subject matter may include any of the optional or preferred features of the other embodiments of the present subject matter. The exemplary embodiments herein disclosed are not intended to be exhaustive or to unnecessarily limit the scope of the present subject matter. The exemplary embodiments were chosen and described to explain the principles of the present subject matter so that others skilled in the art may practice the present subject matter. Having shown and described exemplary embodiments of the present subject matter, those skilled in the art will realize that variations and modifications may be made to affect the described subject matter. Many of those variations and modifications will provide the same result and fall within the spirit of the claimed subject matter. It is the intention, therefore, to limit the subject matter only as indicated by the eventual declaration of scope of the claims.

### REFERENCES

1. E. B. Russo, “Cannabinoids in the management of difficult to treat pain. ”
Therapeutics and Clinical Risk Management, vol. 4, no. 1, pp. 245-259, 2008
2. Breivik H, Collett B, Ventafridda V, Cohen R, Gallacher D. Survey of chronic pain
in Europe: prevalence, impact on daily life, and treatment. Eur J Pain 2006; 10: 287.
3. Goldberg D S, Summer J M. Pain as a global public health priority. BMC Public
Health 2011; 11: 770.
4. Institute of Medicine (IOM). Relieving pain in America: a blueprint for
transforming prevention, care, education, and research. Washington, DC: The National
Academies Press, 2011.
5. Fiorenzani, P., S. Lamponi, A. Magnani, I. Ceccarelli, and A. M. Aloisi. 2014. In
Vitro and In Vivo characterization of the new analgesic combination Beta-
caryophyllene and docosahexaenoic acid. Evid. Based Complement. Alternat. Med.
2014
6. A. L. Klauke, I. Racz, B. Pradier, et al. The cannabinoid CB2 receptor-selective
phytocannabinoid beta-caryophyllene exerts analgesic effects in mouse models of
inflammatory and neuropathic pain Eur Neuropsychopharmacol, 24 (4) (2014), pp.
608-620
7. Wolfe M M, Lichtenstein D R, Singh G. Gastrointestinal toxicity of nonsteroidal
antiinflammatory drugs. New England Journal of Medicine. 1999; 340(24): 1888-1899.
8. Fiorucci S, Antonelli E, Morelli A. Mechanism of non-steroidal anti-inflammatory
drug-gastropathy. Digestive and Liver Disease. 2001; 33(2): S35-S43
9. McPartland, J. M. et al. (2014) Care and Feeding of the Endocannabinoid System: a
Systematic Review of Potential Clinical Interventions That Up-regulate the
Endocannabinoid System. PLoS ONE 9, e89566.)
10. Maderna, P, Godson, C. Lipoxins: Resolutionary Road. British Journal of
Pharmacology (2009)158, 947-959
11. Serhan C N, Brain S D, Buckley C D, Gilroy D W, Haslett C, O'Neill L A, et al.
Resolution of inflammation: state of the art, definitions and terms. FASEB J.
2007; 21: 325-332.)
12. Serhan C N, Chiang N, Van Dyke T E. Resolving inflammation: dual anti-
inflammatory and pro-resolution lipid mediators. Nat Rev. 2008a; 8: 349-361.)
13. https://pubchem.ncbi.nlm.nih.gov/compound/445580#section=Pharmacology-and-
Biochemistry
14. K K Keppel Hesselink J M. Autacoids: A New Fundament for Pain Medicine of the
21th Century. Anaesthesia. Critical Care and Pain Management. 2016; 1: 3-6.
15. Keppel Hesselink J M. Glia as a new target for neuropathic pain, clinical proof of
concept for palmitoylethanolamide, a glia modulator. Anesth Pain Intensive Care
2011; 15: 143-5.
16. O'Sullivan, S. E. (2007). “Cannabinoids go nuclear: evidence for activation of
peroxisome proliferator-activated receptors”. British Journal of Pharmacology. 152 (5):
576-582.
17. Keppel Hesselink, J M (2012). “New Targets in Pain, Non-Neuronal Cells, and the
Role of Palmitoylethanolamide” (review). The Open Pain Journal. 5: 12-23.
18. Jan M. Keppel Hesselink. “Autacoids: A New Fundament for Pain Medicine of the
21th Century”. Anaesthesia, Critical Care and Pain Management 1.1 (2016): 3-6.)
19. Ray W A, Disease C H, Varas-Lorenzo C, Chung C P, Castellsague J, Murray K T, et
al. Cardiovascular risks of nonsteroidal anti-inflammatory drugs in patients after
hospitalization for serious. Circ Cardiovasc Qual Outcomes. 2009
20. Baigent C, Bhala N, Emberson J, Merhi A, Abramson S, Arber N, et al. Vascular
and upper gastrointestinal effects of non-steroidal anti-inflammatory drugs: Meta-
analyses of individual participant data from randomized trials. Lancet.
2013; 382(9894): 769-79.
21. R R Cristino, L.; Bisogno, T.; Di Marzo, V. Cannabinoids and the Expanded
Endocannabinoid System in Neurological Disorders. Nat. Rev. Neurol. 2020, 16, 9-29.
22. V V Zou, S.; Kumar, U. Cannabinoid Receptors and the Endocannabinoid System:
Signaling and Function in the Central Nervous System. Int. J. Mol. Sci. 2018, 19, 833
23. Pacher, P. and Kunos, G. (2013) Modulating the Endocannabinoid System in
Human Health and Disease-Successes and Failures. FEBS J. 280, 1918-1943)
24. Russo, E. B. (2004) Clinical Endocannabinoid Deficiency (CECD): Can This
Concept Explain Therapeutic Benefits of Cannabis in Migraine, Fibromyalgia,
Irritable Bowel Syndrome and Other Treatment-Resistant Conditions?
Neuroendocrinol. Lett. 25, 31-39
25. A Conversation With Dr. Ethan Russo On CBD & Clinical Endocannabinoid
Deficiency, https://www.projectcbd.org/science/cbd-clinical-endocannabinoid-
deficiency-dr-ethan-russo
26. U.S. Pat. No. 6,630,507 Cannabinoids as Antioxidants and Neuroprotectants.
27. Cravatt BF, Giang DK, Mayfield SP, Boger DL, Lerner RA, Gilula NB. Molecular
characterization of an enzyme that degrades neuromodulatory fatty-acid amides.
Nature. 1996; 384: 83-87.
28. Endocannabinoid System . . . What Is It?
(https://www.crescolabs.com/endocannabinoid-system/)
29. National Academies. The Health Effects of Cannabis and Cannabinoids: The
Current State of Evidence and Recommendations for Research. Washington, DC: The
National Academies Press. 2017.
30. What Is Homeostasis? https://www.scientificamerican.com/article/what-is-
homeostasis/.)
31. Silver R J. The Endocannabinoid System of Animals. Animals (Basel). 2019 Sep.
16; 9(9): 686
32. Morena, M.; Patel, S.; Bains, J. S.; Hill, M. N. Neurobiological interactions between
stress and the endocannabinoid system. Neuropsychopharmacology 2016, 41, 80-102.
33. Godlewski, G.; Offertáler, L.; Wagner, J. A.; Kunos, G. (2009). “Receptors for
acylethanolamides-GPR55 and GPR119”. Prostaglandins & Other Lipid Mediators.
89 (3-4): 105-297.
34. Medeiros, R., G. F. Passos, C. E. Vitor, J. Koepp, T. L. Mazzuco, L. F. Pianowski,
et al. 2007. Effect of Two Active Compounds Obtained From the Essential Oil of
Cordia Verbenacea on the Acute Inflammatory Responses Elicited by LPS in the Rat
Paw. Br. J. Pharmacol. 151: 618-627.
35. Chovatiya R and Medzhitov R. (2014) Stress, Inflammation, and Defense of
Homeostasis. Molecular Cell 54
36. Whyte L S, Ryberg E, Sims N A, Ridge S A, Mackie K, Greasley P J, Ross R A,
Rogers M J (September 2009). “The putative cannabinoid receptor GPR55 affects osteoclast
function in vitro and bone mass in vivo”. Proceedings of the National Academy of
Sciences of the United States of America. 106 (38): 16511-6.
37. Keppel Hesselink J M. 2018. Chronic Pain and the Use Of Palmitoylethanolamide.
Journal of the Neurological Sciences. 5(2): 104-248.
38. Roviezzo, F.; Rossi, A., et. al., Palmitoylethanolamide Supplementation during
sensitization Prevents Airway Allergic Symptoms in the Mouse. Front. Pharmacol.
2017, 8, 857.
39. Petrosino S, Schiano Moriello A, Cerrato S, Fusco M, Puigdemont A, De
Petrocellis L, Di Marzo V. The anti-inflammatory mediator palmitoylethanolamide
enhances the levels of 2-arachidonoyl-glycerol and potentiates its actions at TRPV1
cation channels. Br J Pharmacol. 2016 April; 173(7): 1154-62.
40. Placement in Schedule V of Certain FDA-Approved Drugs Containing
Cannabidiol: Final Order. US Drug Enforcement Administration, Department of
Justice. Fed Regist. 2018 Sep. 28; 83(189): 48950-3.
41. Argueta D A, Ventura C M, et al. A Balanced Approach for Cannabidiol Use in
Chronic Pain. Front Pharmacol. 2020; 11: 561
42. Epidiolex approved by FDA
https://www.accessdata.fda.gov/drugsatfda_docs/nda/2018/210365Orig1s000TOC.cfm
43. D'Agostino, G.; Russo, et. al. Palmitoylethanolamide protects against the
amyloid-25-35-induced learning and memory impairment in mice, an experimental
model of Alzheimer disease. Neuropsychopharmacology 2012, 37, 1784-1792.
44. Esposito, E.; Cuzzocre, S. Palmitoylethanolamide in homeostatic and traumatic
central nervous system injuries. CNS Neurol. Disord. Drug Targets 2013, 12, 55-61.
45. Scuderi, C.; Stacca, C.; Valenza, M.; Ratano, P.; Bronzuoli, M. R.; Bartoli, S.;
Steardo, L.; Pompili,
46. De Palma, L.; Santamato, A.; Montillo, V.; Fiore, P. Co-ultra-micronized
palmitoylethanolamide/luteolin treatment as add-on to intensive neuro-rehabilitation in
a young patient with traumatic brain injury. Gazz Med. Ital. Arch. Sci. Med. 2018,
177, 237-242.
47. Petrosino, S.; Di Marzo, V. The pharmacology of palmitoylethanolamide and first
data on the therapeutic efficacy of some of its new formulations. Br. J. Pharmacol.
2017, 174, 1349-1365. therapeutics. Behav Pharmacol. 2021 Apr. 1; 32(2&3): 142-152.
48. Rautiainen, S.; Manson, J. E.; Lichtenstein, A. H.; Sesso, H. D. Dietary supplements
and disease prevention-A global overview. Nat. Rev. Endocrinol. 2016, 12, 407-420.
49. Bronzuoli, M. R.; Facchinetti, R.; et. al. Palmitoylethanolamide Dampens Reactive
Astrogliosis and Improves Neuronal Trophic Support in a Triple Transgenic Model of
Alzheimer's Disease: In Vitro and In Vivo Evidence. Oxid Med. Cell Longev. 2018,
2018, 4720532.
50. Staton C., Hatcher J. P, et al 2008. The Putative Cannabinoid Receptor GPR55
Plays a Role in Mechanical Hyperalgesia Associated With Inflammatory and
Neuropathic Pain. Volume 139; Issue 1; p225-236.)
51. Kandasamy R, Morgan M M. ‘Reinventing the wheel’ to advance the development
of pain E.; Fumagalli, L.; Campolongo, P.; et al. Palmitoylethanolamide controls
reactive gliosis and exerts neuroprotective functions in a rat model of Alzheimer's
disease. Cell Death Dis. 2014, 11, e1419.
52. Piscitelli F, Di Marzo V. “Redundancy” of endocannabinoid inactivation: new
challenges and opportunities for pain control. ACS Chem Neurosci. 2012; 3(5): 356-
363.]
53. Gertsch, J., Leonti, M., et al. (2008) Beta-caryophyllene is a dietary cannabinoid.
Proceedings of the National Academy of Sciences: 105, 26, 9099-9104
54. Franceschi, C., Garagnani, P., Parini, P. et al. Inflammaging: a new immune-
metabolic viewpoint for age-related diseases. Nat Rev Endocrinol 14, 576-590 (2018).
55. Calder, PC. Omega-3 Fatty Acids and Inflammatory Processes. Nutrients.
2010: 2(3): 355-374.
56. Keppel Hesselink J M. Evolution in pharmacologic thinking around the natural
analgesic palmitoylethanolamide: from nonspecific resistance to PPAR-α agonist and
effective nutraceutical. J Pain Research 2013 Aug. 8; 6: 625-34.
57. Ben-Shabat, Shimon; Fride, Ester; Sheskin, Tzviel; Tamiri, Tsippy; Rhee, Man-
Hee; Vogel, Zvi; Bisogno, Tiziana; De Petrocellis, Luciano; Di Marzo, Vincenzo;
Mechoulam, Raphael (July 1998). “An Entourage Effect: Inactive Endogenous Fatty
Acid Glycerol Esters Enhance 2-Arachidonoyl-Glycerol Cannabinoid Activity”.
European Journal of Pharmacology. 353 (1): 23-31.
58. Davis MP, Behm B, Mehta Z, Fernandez C. The Potential Benefits of
Palmitoylethanolamide in Palliation: A Qualitative Systematic Review. Am J Hosp
Palliat Med. 2019; 36(12): 1134-1154.
59. Roncati, L. et al., Micronized/ultramicronized palmitoylethanolamide (PEA) as
natural neuroprotector against COVID-19 inflammation, Prostaglandins & Other Lipid
Mediators, Volume 154, 2021, 106540, ISSN 1098-8823
60. Ceruti S. From astrocytes to satellite glial cells and back: A 25 year-long journey
through the purinergic modulation of glial functions in pain and more. Biochem
Pharmacol. 2021 May; 187: 114397
61. Keppel Hesselink J M, de Boer T, Witkamp R F (2013). “Palmitoylethanolamide:
A Natural Body-Own Anti-Inflammatory Agent, Effective and Safe against Influenza
and Common Cold”. International Journal of Inflammation. 2013: 1-8.
62. Lo Verme, J.; Fu, J.; Astarita, G.; La Rana, G.; Russo, R.; Calignano, A.; Piomelli,
D. (2005). “The nuclear receptor peroxisome proliferator-activated receptor-alpha
mediates the anti-inflammatory actions of palmitoylethanolamide”. Molecular
Pharmacology. 67 (1): 15-19
63. O'Sullivan, S. E.; Kendall, D. A. (2010). “Cannabinoid activation of peroxisome
proliferator-activated receptors: Potential for modulation of inflammatory disease”.
Immunobiology. 215 (8): 611-616.
64. Ryberg E, Larsson N, Sjogren S, Hjorth S, Hermansson N O, Leonova J, Elebring
T, Nilsson K, Drmota T, Greasley P J (December 2007). “The orphan receptor GPR55 is a
novel cannabinoid receptor”. British Journal of Pharmacology. 152 (7): 1092-101.
doi:10.1038/sj.bjp.0707460.
65. Lauckner J E, Jensen J B, Chen H Y, Lu H C, Hille B, Mackie K (February 2008).
“GPR55 is a cannabinoid receptor that increases intracellular calcium and inhibits M
current”. Proceedings of the National Academy of Sciences of the United States of
America. 105 (7): 2699-704.
66. Jonsson, K. O.; Vandevoorde, S. V.; Lambert, D. M.; Tiger, G.; Fowler, C. J.
(2001). “Effects of homologues and analogues of palmitoylethanolamide upon the
inactivation of the endocannabinoid anandamide”. British Journal of Pharmacology.
133 (8): 1263-1275.
67. Ho, W. S.; Barrett, D. A.; Randall, M. D. (2008). “‘Entourage’ effects of N-
palmitoylethanolamide and N-oleoylethanolamide on vasorelaxation to anandamide
occur through TRPV1 receptors”. British Journal of Pharmacology. 155 (6): 837-846.
68. De Filippis, D.; d'Amico, A.; Cipriano, M.; Petrosino, S.; Orlando, P.; Di Marzo,
V.; Iuvone, T. (2010). “Levels of endocannabinoids and palmitoylethanolamide and
their pharmacological manipulation in chronic granulomatous inflammation in rats”.
Pharmacological Research. 61 (4): 321-328.
69. Atakan Z. Cannabis, a complex plant: different compounds and different effects on
individuals. Therapeutic Advances in Psychopharmacology. December 2012: 241-254.
70. Calignano a, L. R. G. (2001). “ Antinociceptive activity of the endogenous fatty
acid amide, palmitoylethanolamide”. Eur J Pharmacol. 419 (2-3): 191-198.
71. Koch, M.; Kreutz, S.; Böttger, C.; Benz, A.; Maronde, E.; Ghadban, C.; Korf, H.
W.; Dehghani, F. (2010). “Palmitoylethanolamide Protects Dentate Gyrus Granule
Cells via Peroxisome Proliferator-Activated Receptor-Alpha”. Neurotoxicity Research.
19 (2): 330-340.
72. Lambert D M, Vandevoorde S, Diependaele G, Govaerts S J, Robert A R (2001).
“Anticonvulsant activity of Npalmitoylethanolamide, a putative endocannabinoid, in
mice”. Epilepsia. 42 (3): 321-7.
73. Keppel Hesselink J M. Evolution in pharmacologic thinking around the natural
analgesic palmitoylethanolamide: from nonspecific resistance to PPAR-α agonist and
effective nutraceutical. J Pain Res. 2013 Aug. 8; 6: 625-34.
74. Nestmann ER Safety of micronized palmitoylethanolamide (microPEA): lack of
toxicity and genotoxic potential. Food Sci Nutr. 2017 March; 5(2): 292-309
75. Skaper SD, Facci L, et. al., N-Palmitoylethanolamide and Neuroinflammation: a
Novel Therapeutic Strategy of Resolution. Molecular Neurobiology 52. 1034-1042
(2015)
76. Alhouayek M. et al, Controlling 2-arachidonoylglycerol metabolism as an anti-
inflammatory strategy. Drug Discovery Today Volume 19, Number 3 2014 p 295-
304.
77. Skaper SD, Facci L, and Giusti P. Glia and mast cells as targets for
palmitoylethanolamide, an anti-inflammatory and neuroprotective lipid mediator. Mol
Neurobiol. 2013 October; 48(2): 340-52
78. Keppel Hesselink J M. Chronic Pain and the Use of Palmitoylethanolamide. Austin
J Neurol Discord Epilepsy. 2018; 5(2): 1042.
79. Petrosino S, Schiano Moriello A. Palmitoylethanolamide: A Nutritional Approach
to Keep Neuroinflammation within Physiological Boundaries-A Systematic Review.
Int J Mol Sci. 2020 Dec. 15; 21(24): 9526.
80. RUSSO, E. Cannabinoids in the management of difficult to treat pain. Ther Clin
Risk Manag. 2008 February; 4(1): 245-259.
81. Choi CI. Astaxanthin as a Peroxisome Proliferator-Activated Receptor (PPAR)
Modulator: Its Therapeutic Implications. Mar Drugs. 2019 Apr. 23; 17(4): 242.
82. Kishimoto, Y.; Yoshida, H.; Kondo, K. Potential Anti-Atherosclerotic Properties
of Astaxanthin. Mar. Drugs 2016, 14, 35,
83. Kim, S. H.; Lim, J. W.; Kim, H. Astaxanthin Inhibits Mitochondrial Dysfunction
and Interleukin-8 Expression in Helicobacter pylori-Infected Gastric Epithelial Cells.
Nutrients 2018, 10, 1320.
84. Mirza, A. Z.; Althagafi, I. I.; Shamshad, H. Role of PPAR receptor in different
diseases and their ligands: Physiological importance and clinical implications. Eur. J.
Med. Chem. 2019, 166, 502-513.
85. Guerin, M.; Huntley, M. E.; Olaizola, M. Haematococcus astaxanthin: Applications
for human health and nutrition. Trends Biotechnol. 2003, 21, 210-216.
86. Ji, R.-R.; Chamessian, A.; Zhang, Y.-Q. Pain Regulation by Non-neuronal Cells
and Inflammation. Science 2016, 354, 572-577.
87. Boche D, Perry VH, Nicoll JA Review: Activation patterns of microglia and their
identification in the human brain. Neuropathol Appl Neurobiol. 2013 Feb; 39(1): 3-18.
88. Bordon Y Neuroinflammation: Inflammatory brain drain. Nat Rev Immunol. 2013
February; 13(2): 69
89. Ren K, Dubner R Interactions between the immune and nervous systems in pain.
Nat Med. 2010 November; 16(11): 1267-76.
90. Dong H, Zhang X, Qian Y Mast cells and neuroinflammation. Med Sci Monit
Basic Res. 2014 Dec. 21; 20: 200-6.
91. Review: activation patterns of microglia and their identification in the human
brain. Boche D, Perry V H, Nicoll J A Neuropathol Appl Neurobiol. 2013 February; 39(1): 3-
18.
92. Nelissen S, Lemmens E, Geurts N, Kramer P, Maurer M, Hendriks J, Hendrix S.
The role of mast cells in neuroinflammation. Acta Neuropathol. 2013 May;
125(5): 637-50.
93. Dong H, Zhang X, Qian Y Mast cells and neuroinflammation. Med Sci Monit
Basic Res. 2014 Dec. 21; 20( ): 200-6.
94. Ji RR, Xu ZZ, Gao YJ Emerging targets in neuroinflammation-driven chronic pain.
Nat Rev Drug Discov. 2014 July; 13(7): 533-48.
95. Neuroinflammation, microglia and mast cells in the pathophysiology of
neurocognitive disorders: a review. Skaper SD, Facci L, Giusti PCNS Neurol Disord
Drug Targets. 2014; 13(10): 1654-66.),
96. Britti, D., Crupi, R., Impellizzeri, D. et al. A novel composite formulation of
palmitoylethanolamide and quercetin decreases inflammation and relieves pain
in inflammatory and osteoarthritic pain models. BMC Vet Res 13, 229 (2017).
https://doi.org/10.1186/s12917-017-1151-z
97. Microglia change from a reactive to an age-like phenotype with the time in culture.
Caldeira C, Oliveira AF, Cunha C, Vaz AR, Falcao AS, Fernandes A, Brites D Front
Cell Neurosci. 2014; 8( ): 152.
98. Mast cells in neuroinflammation and brain disorders. Hendriksen E, van Bergeijk
D, Oosting R S, Redegeld F A Neurosci Biobehav Rev. 2017 August; 79( ): 119-133.
99. Refolo, V.; Stefanova, N. Neuroinflammation and Glial Phenotypic Changes in
Alpha-Synucleinopathies. Front. Cell. Neurosci. 2019, 13, 263.
100. Skaper, S. D.; Facci, L.; Zusso, M.; Giusti, P. An Inflammation-Centric View of
Neurological Disease: Beyond the Neuron. Front. Cell. Neurosci. 2018, 12, 72.
101. Skaper, S. D.; Facci, L. Mast cell-glia axis in neuroinflammation and therapeutic
potential of the anandamide congener palmitoylethanolamide. Philos. Trans. R. Soc.
Lond. B Biol. Sci. 2012, 367, 3312-3325.
102. Keppel Hesselink J M. Chronic Pain and Use of Palmitoylethanolamide. Austin J
Neurol Disord Epilepsie-Volume 5 Issue 2-2018 ISSN 2472-3711].
103. Henstridge C. M. Balenga N. A. Ford L. A. Ross R. A. Waldhoer M. Irving A. J.
FASEB J. 2009; 23: 183-193.
104. Gabrielsson, L.; Mattson, S.; Fowler, C. J. Palmitoylethanolamide for the
treatment of pain: Pharmacokinetics, safety and efficacy. Br. J. Clin. Pharmacol. 2016,
82, 932-942.
105. Skaper, S. D.; Facci, L.; Giusti, P. Mast cells, glia and neuroinflammation:
Partners in crime? Immunology 2014, 141, 314-327.
106. Skaper, S. D.; Facci, L. Mastcell-glia axis in neuroinflammation and therapeutic
potential of the anandamide congener palmitoylethanolamide. Philos. Trans. R. Soc.
Lond. B Biol. Sci. 2012, 367, 3312-3325.
107. FDA Public Notice FDA Regulation of Cannabis and Cannabis-Derived
Products, Including Cannabidiol (CBD) https://www.fda.gov/news-events/public-
health-focus/fda-regulation-cannabis-and-cannabis-derived-products-
includingcannabidiol-cbd
108. Clayton, P.; Hill, M.; Bogoda, N.; Subah, S.; Venkatesh, R. Palmitoylethanolamide:
A Natural Compound for Health Management. Int. J. Mol. Sci. 2021, 22, 5305.
109. G.R.A.S. Federal Register Notice-the GRAS Final Rule (81 FR 54960-
Aug. 17, 2016) https://www.federalregister.gov/documents/2016/08/17/2016-
19164/substances-generally-recognized-as-safe
110. Russo, E. et al. (2002) in Search of Plants, Other Than Cannabis Sativa, With
Cannabinoid Receptor Activity. In Symposium on the Cannabinoids. International
Cannabinoid Research Society, pp. 46.
111. Yatoo M I et al., Anti-Inflammatory Drugs and Herbs with Special Emphasis on
Herbal Medicines for Countering Inflammatory Diseases and Disorders-A Review.
Recent Patents on Inflammation & Allergy Drug Discovery. 12 (1)
112. Merskey H, Bogduk N. Classification of chronic pain. 2nd ed. Seattle: IASP
Press, 1994. p. 1.
113. Bonica J J. The management of pain. Philadelphia: Lea & Febiger, 1953.
114. Julius D. TRP Channels and Pain The Annual Review of Cell and Developmental
Biology. 2013. 29: 355-84
115. Shenglong Zou and Ujendra Kumar. Cannabinoid Receptors and the
Endocannabinoid System: Signaling and Function in the Central Nervous System. Int.
J. Mol. Sci. 2018, 19(3), 833.
116. Fernandes E S, Passos G F, Medeiros R, da Cunha F M, Ferreira J, Campos M M, et
al. (2007). Anti-inflammatory effects of compounds alpha-humulene and (—)-trans-
caryophyllene isolated from the essential oil of Cordia verbenacea. Eur J Pharmacol
569: 228-236.
117. Russo, Ethan B (2011). “Taming THC: Potential Cannabis Synergy and
Phytocannabinoid-Terpenoid Entourage Effects”. British Journal of Pharmacology.
163 (7): 1344-1364.
118. Davis A, Robson J. The dangers of NSAIDs: Look both ways. Br J Gen Pract.
2016; 66(645): 172-173.)
119. Ferrero-Miliani L, Nielsen O H, Andersen P S, Girardin S E; Nielsen; Andersen;
Girardin (February 2007). “Chronic inflammation: importance of NOD2 and NALP3
in interleukin-1beta generation”. Clin. Exp. Immunol. 147 (2): 227-35.
120. Vetter G, Brüggemann G, Lettko M, et al.. Shortening diclofenac therapy by B
vitamins. Results of a randomized double-blind study, diclofenac 50 mg versus
diclofenac 50 mg plus B vitamins, in painful spinal diseases with degenerative
changes. Z Rheumatol 1988; 47(5): 351-62.
121. Ponce-Monter HA , Ortiz M I, Garza-Hernández A F, Monroy-Maya R, Soto-Ríos,
M, Carrillo-Alarcón L, et al. Effect of diclofenac with B vitamins on the treatment of
acute pain originated by lower-limb fracture and surgery. Pain Res Treat. 2012;
2012: 104782.
122. Otten J J, Pitzi Hellwing J, Meyers L D (2009) Dietary Reference Intakes:
Essential Guide to Nutrient Requirements. National Academies Press, Washington,
DC.
123. Munvar Miya Shaik, Siew Hua Gan, “Vitamin Supplementation as Possible
Prophylactic Treatment against Migraine with Aura and Menstrual Migraine”, BioMed
Research International, vol. 2015, Article ID 469529, 10 pages, 2015.
124. Calderón-Ospina C A, Nava-Mesa M O. B Vitamins in the nervous system:
Current knowledge of the biochemical modes of action and synergies of thiamine,
pyridoxine, and cobalamin. CNS Neurosci Ther. 2020 January; 26(1): 5-13.
125. Shideler C. Vitamin B6: an overview. Am J Med Technol. 1983; 49(1): 17-22
126. Geller, Mauro & Oliveira, Lisa & Nigri, Rafael & Mezitis, Spyros & Ribeiro,
Marcia & Fonseca, Adenilson & Guimaraes, Oscar & Kaufman, Renato & Wajnsztajn,
Fernanda. (2017). B Vitamins for Neuropathy and Neuropathic Pain. Vitamins &
Minerals. 06.10.4172/2376-1318.1000161.
127. Garg S, Syngle A, Vohra K. Efficacy and tolerability of advanced glycation end-
products inhibitor in osteoarthritis: a randomized, double-blind, placebo-controlled
study. Clin J Pain. 2013; 29(8): 717-24.
128. Mibielli M A, Geller M, Cohen J C, Goldberg S G, Cohen M T, Nunes C P, et al.
Diclofenac plus B vitamins versus diclofenac monotherapy in lumbago: the DOLOR
study. Curr Med Res Opin. 2009; 25(11): 2589-99.
129. Chapkin, R. S., W. Kim, J. R. Lupton, and D. N. McMurray. 2009. Dietary
docosahexaenoic and eicosapentaenoic acid: emerging mediators of inflammation.
Prostaglandins Leukot. Essent. Fatty Acids 81: 187-191.
130. Serhan C N, Hong S, Gronert K, Colgan S P, Devc- hand P R, Mirick G,
Moussignac R-L. Resolvins: a family of bioactive products of omega-3 fatty acid
transformation circuits initiated by aspirin treat- ment that counter pro-inflammation
signals. J Exp Med. 2002; 196: 1025-37
131. Varga, et al, (2017) Beta-Caryophyllene Protects Against Alcoholic
Steatohepatitis by Attenuating Inflammation and Metabolic Dysregulation in Mice.
British Journal of Pharmacology (2018) 175 320-334
132. Javid H, Azimullah S, Haque M E and Ojha S K (2016) Cannabinoid Type 2 (CB2)
Receptors Activation Protects against Oxidative Stress and Neuroinflammation
Associated Dopaminergic Neurodegeneration in Rotenone Model of Parkinson's
Disease. Front. Neurosci. 10: 321.
133. Federal Trade Commission Announcement. FTC Announces Crackdown on
Deceptively Marketed CBD Products. Dec. 17, 2020 (https://www.ftc.gov/news-
events/press-releases/2020/12/ftc-announces-crackdown-deceptively-marketed-
cbdproducts)
134. De Filippis, D.; d'Amico, A.; Cipriano, M.; Petrosino, S.; Orlando, P.; Di Marzo,
V.; Iuvone, T.; Iuvone, T. (2010). “Levels of endocannabinoids and
palmitoylethanolamide and their pharmacological manipulation in chronic
granulomatous inflammation in rats”. Pharmacological Research. 61 (4): 321-328.
135. Astarita, G., Geaga, J., Ahmed, F., and Piomelli, D. (2009) Targeted lipidomics
as a tool to investigate endocannabinoid function. Int. Rev. Neurobiol. 85, 35-55.
136. Philip C. Calder Omega-3 Fatty Acids and Inflammatory Processes. Nutrients.
2010 Mar; 2(3): 355-374.
137. Ulbricht, CE, et al. (2014). An Evidence-Based Systematic Review of
Chlorophyll by the Natural Standard Research Collaboration. Journal of Dietary
Supplements, 2014Stillwell W, Wassall S R. Docosahexaenoic acid: membrane
properties of a unique fatty acid. Chem Phys Lipids. 2003; 126: 1-27.
138. Stillwell W, Wassall S R. Docosahexaenoic acid: membrane properties of a
unique fatty acid. Chem Phys Lipids. 2003; 126: 1-27.
139. Jolayemi and Ojewole (2913) Comparative Anti-Inflammatory Properties of
Capsaicin and Ethyl-Acetate Extract of Capsicum Frutescens in Rats. Afr Health Sci.
2013 June; 13(2): 357-361.
140. Wei & Manickam (2012) Response Surface Methodology, an Effective Strategy
in the Optimization of the Generation of Curcumin-Loaded Micelles. Asia-Pacific
Journal of Chemical Engineering Volume 7, Issue S1
141. Yousef S A, et al. (2019) Mechanistic Evaluation of Enhanced Curcumin Delivery
through Human Skin In Vitro from Optimized Nano-emulsion Formulations Fabricated
with Different Penetration Enhancers, Pharmaceutics 2019, 11(12), 639
142. FDA's SCOGS Database; Silicon Dioxides, Report No. 61, 1979.; ID Code:
14808-60-7;
http://www.accessdata.fda.gov/scripts/fcn/fcnDetailNavigation.cfm?rpt=scogsListing&
id=276; accessed Aug. 12, 2011)]
143. Trice J M, Pinals R S. DimethylSulfoxide: a Review of its Use in the Rheumatic
Disorders. Seminars in Arthritis and Rheumatism. 1985; 15: 45-60.
144. Muir M. DMSO. Alternative and Complementary Therapies. 1996; 2: 230-235.
10.1089/act.1996.2.230
145. Kathrin-Maria Roy “Sulfones and Sulfoxides” in Ullmann's Encyclopedia of
Industrial Chemistry, 2002, Wiley-VCH, Weinheim. doi:10.1002/14356007.a25_487
146. Uraz S, et. al., N-acetylcysteine expresses powerful anti-inflammatory and
antioxidant activities resulting in complete improvement of acetic acid-induced colitis
in rats. Scandinavian Journal of Clinical and Laboratory Investigation. 2013
February; 73(1): 61-6.
147. Jonscher, K. R.; Chowanadisai, W.; Rucker, R. B. Pyrroloquinoline-Quinone Is
More Than an Antioxidant: A Vitamin-like Accessory Factor Important in Health and
Disease Prevention. Biomolecules Biomolecules 2021, 11, 1441.)
148. Biswal S. Oxidative stress and astaxanthin: The novel supernutrient carotenoid.
Int J Health Allied Sci. 2014 Jul. 1; 3(3): 147.
149. Forman, Henry Jay; Zhang, Hongqiao; Rinna, Alessandra (2009). “Glutathione:
Overview of its protective roles, measurement, and biosynthesis”. Molecular Aspects
of Medicine. 30 (1-2): 1-12.)

What is claimed:

1. A method of helping to replenish levels of endogenous palmitoylethanolamide (PEA) and docosahexaenoic acid (DHA), restoring their protective, anti-inflammatory, and analgesic effects, and protecting the health of a mammal in need thereof, comprising a synergistic supplemental daily dosage form containing exogenous palmitoylethanolamide (PEA) in an ultra-micronized form, docosahexaenoic acid (DHA) and beta-caryophyllene (BCP); wherein palmitoylethanolamide (PEA) is contained in an amount between 25 mg and 1200 mg per dosage, preferably 50 mg to 1000 mg, and more preferably 50 mg to 600 mg, and not exceeding 3600 mg per dose; wherein beta-caryophyllene (BCP) is contained in an amount between 10 mg and 500 mg per dosage, preferably 15 mg to 300 mg, and more preferably 20 mg to 150 mg, and not exceeding 750 mg mg per dose; wherein docosahexaenoic acid (DHA) is contained in an amount between 0.5 mg and 250 mg per dosage, preferably 1 mg to 100 mg, and more preferably 5 mg to 50 mg, and not exceeding 300 mg per dose; wherein the supplemental daily dosage form has palmitoylethanolamide in a weight percentage between about 2 and about 85%, preferably between 5 and 30%, and more preferably between 7 and 15%, beta-caryophyllene (BCP) in a weight percentage between about 0.1 and about 20%, preferably between 2 and 11%, and more preferably between 5 and 7.5%, and docosahexaenoic acid (DHA) in a weight percentage between about 0.001 and about 15%, preferably between 0.01 and 5%, and more preferably between 0.01 and 2.5%.

2. The method of claim 1, wherein the pharmaceutical daily dosage form is an oral, rectal, topical, or transdermal dosage form, comprising one or more of cream, lotion, balm, or ointment.

3. The method of claim 1, wherein the pharmaceutical daily dosage form further comprises one or more compounds with anti-inflammatory and/or antioxidant activity in a total weight percentage ranging between 1% and about 20%.

4. The method of claim 3, wherein the one or more compounds with anti-inflammatory activity is selected from the group comprising plant derived terpenes/terpenoids, carotenoids, polyphenols, B vitamins, and combinations thereof.

5. The method of claim 4, wherein the one or more compounds with anti-inflammatory activity, includes humulene from about 50 mg to about 3500 mg, but preferably from 75 mg to 750 mg, and more preferably 100 mg to 150 mg.

6. The method of claim 3, wherein the one or more compounds with anti-inflammatory activity is selected from the group consisting of polyphenols or carotenoids.

7. The method of claim 6, wherein the one or more compounds with anti-inflammatory activity, comprises a polyphenol selected from the group consisting of Flavonoids, Polyphenolic amides, other polyphenols, and combinations thereof.

8. The method of claim 7, wherein the flavonoid is quercetin, the polyphenolic amide is a capsaicinoid, and the other polyphenol is curcumin.

9. The method of claim 3, wherein the synergistic supplemental daily dosage form further comprises: an active dosage of palmitoylethanolamide in an amount of not exceeding about 3600 mg/day; an active dosage not exceeding 0.1 mL/kg of beta-caryophyllene (BCP) and docosahexaenoic acid (DHA) in a total weight percentage ranging between 0 and about 20% but not exceeding about 10 mg/kg; and an active dosage of the one or more compounds with an anti-inflammatory activity in a total weight percentage ranging between 0 and about 20% such that the synergistic supplemental daily dosage form is one of a dosage tablet, a capsule, or liquid form of a supplemental composition.

10. The method of claim 3, wherein the one or more compounds with the anti-inflammatory activity is co-micronized with the palmitoylethanolamide.

11. The method of claim 1, wherein the neuropathic pain results from a disease selected from the group consisting of both acute and chronic painful central and peripheral neuropathies; migraines; fibromyalgia; pain associated with vertebral column and spinal cord diseases of traumatic, dysmetabolic and degenerative origin; acute and/or chronic pain associated with diseases in the pelvic area; Irritable Bowel Syndrome; pain associated with traumatic and degenerative joint diseases; pain associated with arthritic diseases, and any combination thereof,

12. The method of claim 1, wherein the supplemental daily dosage form is for human or veterinary use.

13. The method of claim 1, wherein the palmitoylethanolamide is in ultra-micronized form (um PEA) with a particle size ranging between about 0.8 and about 10 microns.

14. A method of treating neuropathic and inflammatory pain in a mammal, comprising:
administering to a mammal in need thereof a dosage form selected from the group consisting of a chew, gummy, patty, cookie, freeze-dried food product, suppository, powder, tablet, capsule, or a liquid or non-solid form of a synergistic supplemental composition, wherein each dosage form of the supplemental composition comprises:
palmitoylethanolamide (PEA) in a weight percentage between about 5 and about 100%, more preferably between about 5 and 15%, and more preferably between about 5 and 10%;
beta-caryophyllene (BCP) in a weight percentage between about 1 and about 10%, more preferably between about 2.5 and 7.5%, and more preferably between about 5 and 7.5%;
and docosahexaenoic acid (DHA) in a weight percentage between about 0.001 and about 2.5%, and more preferably between about 0.01 and 2%; and one or more compounds with anti-inflammatory activity in a total weight percentage ranging between 0 and about 20%.

15. The method of claim 14, wherein said palmitoylethanolamide is in non-micronized form with a particle size ranging between about 100 μm and about 2,000 μm, in micronized form (mPEA) with a particle size ranging between about 2 and about 10 μm, or in ultra-micronized form (umPEA) with a particle size ranging between about 0.8 and about 10 μm, or in a mixture of such forms.

16. The method of claim 14, wherein said beta-caryophyllene (BCP) and docosahexaenoic acid (DHA) is in a miscible form or dispersion with the intent of improving ease of use and bioavailability.

17. The method of claim 2, wherein the supplemental daily dosage form is topical or transdermal dosage form.

18. The method of claim 17, wherein the topical dosage form is selected from the group consisting of a transdermal patch, a cream, a lotion, an ointment, a balm, a spray, a liniment, and a powder.

19. The method of claim 1, wherein the pharmaceutical daily dosage form further comprises one or more of cannabidiol (CBD), cannabidiolic acid (CBDA), tetrahydrocannabinol (THC) and tetrahydrocannabinolic acid (THCA).
